(19) United States
(12) Patent Application Publication
(10) Pub. No.: US 2008/0139623 A1
(43) Pub. Date: Jun. 12, 2008

(54) Amorphous and crystalline forms of pantoprazole magnesium salt

(76) Inventors: Lilach Hedvati, Doar Na Hefer (IL); Tamas Koltai, Netanya (IL)

Correspondence Address: KENYON & KENYON LLP, ONE BROADWAY, NEW YORK, NY, 10004, US

(21) Appl. No.: 11/818,064

(22) Filed: Jun. 12, 2007

Related U.S. Application Data
(60) Provisional application No. 60/813,176, filed on Jun. 12, 2006.
(60) Provisional application No. 60/833,615, filed on Jul. 26, 2006.
(60) Provisional application No. 60/858,164, filed on Nov. 8, 2006.
(60) Provisional application No. 60/873,674, filed on Dec. 7, 2006.
(60) Provisional application No. 60/926,281, filed on Apr. 25, 2007.

(51) Int. Cl. A61K 31/4439; C07D 401/04; A61P 1/00

(52) U.S. Cl. 514/338; 546/273.7

(57) ABSTRACT

Provided are amorphous and crystalline forms of pantoprazole magnesium salt and processes for their preparation.

### CROSS-REFERENCE TO RELATED APPLICATIONS

This application claims the benefit of priority to U.S. provisional application Ser. Nos. 60/813,176, filed Jun. 12, 2006; 60/833,615, filed Jul. 26, 2006; 60/858,164, filed Nov. 8, 2006; 60/873,674, filed Dec. 7, 2006; 60/926,281, filed Apr. 25, 2007, all of which are hereby incorporated by reference.

### FIELD OF THE INVENTION

The invention encompasses amorphous and crystalline forms of pantoprazole magnesium salt and processes for their preparation.

### BACKGROUND OF THE INVENTION

Pantoprazole magnesium salt (“PNT-Mg”) has the chemical name 5-difluoromethoxy-2-[[3,4-dimethoxy-2-pyridinyl)methyl]sulfinyl]-1H-benzimidazole magnesium salt and the following chemical structure:

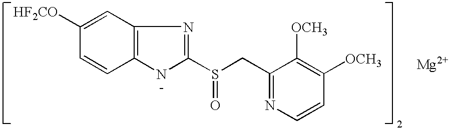

Pantoprazole is a gastric acid secretion inhibitors and are typically used as anti-ulcer agents. Pantoprazole is currently marketed by Altana under the trade name PANTOLOC® in the form of the sodium sesquihydrate salt.

U.S. Pat. No. 4,758,579 (“'579 patent”) refers to a class of fluoroalkoxy-substituted benzimidazoles, which includes pantoprazole and salts thereof. '579 patent, col. 2, 11. 1-33; col. 5, 11. 23-24. The '579 patent states that preferred salts of sulfoxide compounds falling within the class of fluoroalkoxy-substituted benzimidazoles, such as pantoprazole, are basic salts including sodium, potassium, calcium, and aluminum salts. Id. at col. 3,11, 14-19.

Magnesium salts of pantoprazole have also been prepared. For example, U.S. Pat. No. 6,124,464 (“'464 patent”) refers to magnesium salts of a class of substituted sulfinyl heterocyclic compounds, which includes pantoprazole magnesium. '464 patent, col. 2, 1. 41 to col. 3, 1. 52. The '464 patent also exemplifies a process for preparing the pantoprazole magnesium salt. Id. at col. 9, 11. 45-60 (example 10). The Applicants repeated example 10 of the '464 patent and obtained pantoprazole magnesium dihydrate, herein denominated Form A. See Comparative Example 15 below. Crystalline magnesium Form A is characterized by a powder X-ray diffraction pattern having peaks at about 5.8, 14.9, 16.0, 16.6, and 18.3±0.2° 2θ; a powder X-ray diffraction pattern having peaks at about 5.8, 14.9, 16.0, 16.6, 18.3, 10.8, 17.0, 23.3, 25.0, and 25.9±0.2° 2θ; a powder X-ray diffraction pattern substantially as depicted in FIG. 29, an infrared spectrum having peaks at about 3270, 2990, 1592, 1427, 1073, 1037 and 825±2 cm⁻¹; an infrared spectrum substantially as depicted in Figure FIG. 13; a SSNMR spectrum substantially as depicted in FIG. 28. Crystalline pantoprazole magnesium Form A is a dihydrate, having a water content of about 4.4-4.7% by weight.

In addition, U.S. Pat. Nos. 6,410,569 (“'569 patent”) and 6,686,379 (“'379 patent”) refer to pantoprazole magnesium dihydrate and processes for its preparation. '569 patent, col. 2, 1.43 to col. 4, 1.5; '379 patent, col. 2, 1.45 to col. 4, 1.3.

Pharmaceutical solids may exist in different physical forms, including amorphous and crystalline forms. An amorphous form consists of a disordered arrangement of molecules, and does not possess a distinguishable crystal lattice. A crystalline form consists of an ordered arrangement of molecules in a repeating pattern to form a lattice.

When a pharmaceutical solid can exist in two or more crystalline forms that have different arrangements and/or conformations of the molecules in the crystalline lattice, it is said to exhibit polymorphism and the different crystalline forms are called polymorphs. Polymorphs of a pharmaceutical solid may have different physical and solid-state chemical (reactivity) properties. Polymorphs differ in internal solid-state structure and, therefore, possess different chemical and physical properties, including packing, thermodynamic, spectroscopic, kinetic, interfacial and mechanical properties. These properties can have a direct impact on drug product quality/performance, including stability, dissolution, and bioavailability.

The preparation of amorphous forms on an industrial scale is often problematic. Many processes used to prepare amorphous form of an active pharmaceutical ingredient (“API”) are not suitable for industrial scale. For example, to obtain amorphous form of an API by solidification of melt, the API has to be heated beyond its melting point, which may require expenditure of much energy, particularly when the API has a high melting point. Further, the high temperatures may chemically damage the API.

Therefore, there is a need in the art for an amorphous form of pantoprazole magnesium, as well as an industrially feasible process for its preparation. There is also a need in the art for additional crystalline forms of pantoprazole magnesium.

### SUMMARY OF THE INVENTION

In one embodiment, the invention encompasses amorphous pantoprazole magnesium. Also provided is substantially amorphous pantoprazole magnesium, which contains not more than 10% weight percent crystalline pantoprazole magnesium as measured by area percentage using XRD.

In one embodiment, the invention encompasses amorphous pantoprazole magnesium having not more than about 10% weight percent, preferably not more than about 5% weight percent, and more preferably not more than about 1% weight percent of crystalline pantoprazole magnesium dihydrate Form A or Form C as measured by area percentage using XRD.

The invention also encompasses a process for preparing the amorphous pantoprazole magnesium comprising: dissolving pantoprazole magnesium in a solvent selected from the group consisting of at least one of methanol and ethanol; and removing the solvent to obtain the amorphous pantoprazole magnesium.

In another embodiment, the invention encompasses a crystalline form of pantoprazole magnesium (designated as Form C) characterized by at least one of: a powder X-ray diffraction pattern having peaks at about 6.0, 16.0, 19.0 and 19.6±0.2° 2θ; a powder X-ray diffraction pattern having peaks at about 6.0, 16.0, 19.0, 19.6 and 23.3±0.2° 2θ; a powder X-ray diffraction pattern having peaks at about 16.0, 16.6, 18.3, 19.0 and 19.6±0.2° 2θ; a powder X-ray diffraction pattern substantially as depicted in FIG. 5; a PXRD pattern having peak position differences between the peak at 15.0±0.4° 2θ of about 1.0, 1.6, 3.3, 4.0 and 4.6±0.10° 2θ; an infrared spectrum having bands at about 3275, 2991, 1593, 1428, 1074, 1035 and 827±2 cm⁻¹; an IR spectrum substantially as depicted in FIG. 14. Crystalline pantoprazole magnesium form C may be a dihydrate and may contains about 4.7% of water by weight.

The invention also encompasses a process for preparing the crystalline pantoprazole magnesium Form C comprising: combining pantoprazole magnesium with methanol, ethanol, methyl-t-butyl ether, ethyl acetate and mixtures thereof to obtain a slurry of the crystalline pantoprazole magnesium Form C; heating and isolating the crystalline pantoprazole magnesium Form C from the slurry.

In another embodiment, the invention encompasses a crystalline form of pantoprazole magnesium (designated as Form E) characterized by at least one of: a powder X-ray diffraction pattern having peaks at about 5.6, 13.1, and 16.6±0.2° 2θ a powder X-ray diffraction pattern having peaks at about 5.6, 13.1, 16.6, and 22.3±0.2° 2θ; a powder X-ray diffraction pattern having peaks at about 5.6, 12.5, 13.1, and 16.6±0.2° 2θ; a powder X-ray diffraction pattern substantially as depicted in FIG. 7; an infrared spectrum having bands at about 3532, 1664, 1412, 1388, 1002, and 883±2 cm⁻¹; an IR spectrum substantially as depicted in FIG. 15; a solid state ¹³C NMR spectrum having signals at about 102.5, 118.3, 143.8 and 157.7±0.2 ppm; a SSNMR spectrum substantially as depicted in FIG. 19; a the solid state ¹³C NMR spectrum having chemical shift differences between the signal exhibiting the lowest chemical shift and another in the chemical shift range of 100 to 200 ppm of about 0, 15.8, 41.3 and 55.2±0.1 ppm. Form E is a hemipentahydrate and contains about 5.1-6.2% of water by weight.

The invention also encompasses a process for preparing the crystalline pantoprazole magnesium Form E comprising: a) combining pantoprazole free acid, a source of magnesium selected from the group consisting of Mg, Mg(OCH₂CH₃)₂, and Mg(OCH₃)₂, methanol and water to obtain a mixture; b) heating the mixture; c) cooling the mixture to precipitate the crystalline pantoprazole magnesium Form E; and d) isolating the precipitated crystalline pantoprazole magnesium Form E from the mixture.

In another embodiment, the invention encompasses a crystalline form of pantoprazole magnesium (designated as Form F) characterized by at least one of: a powder X-ray diffraction pattern having peaks at about 6.9, 8.9, 9.7, 12.4, and 14.0±0.2° 2θ; a powder X-ray diffraction pattern having peaks at about 6.9, 8.9, 9.7, 12.4, 14.0, 16.9, 17.2, and 22.5±0.2° 2θ; a powder X-ray diffraction pattern substantially as depicted in FIG. 8 or 9; an infrared spectrum having bands at about 3657, 2982, 1587, 1408, 1176, 1156, and 1069±2 cm⁻¹; an IR spectrum substantially as depicted in FIG. 16; a solid state ¹³C NMR spectrum having signals at about 106.1, 142.2, 144.0, and 160.2±0.2 ppm; a SSNMR spectrum substantially as depicted in FIG. 20; a solid state ¹³C NMR spectrum having chemical shift differences between the signal exhibiting the lowest chemical shift and another in the chemical shift range of 100 to 200 ppm of about 0, 36.1, 37.9 and 54.1±0.1 ppm, Form F may be a dimethanolate and contains about 7% methanol by weight.

The invention also encompasses a process for preparing the crystalline pantoprazole magnesium Form F comprising: a) combining pantoprazole free acid, a source of magnesium selected from the group consisting of Mg, Mg(OCH₃)₂ and Mg(OCH₂CH₃)₂, and methanol to obtain a mixture; b) heating the mixture; c) cooling the mixture to precipitate the crystalline pantoprazole magnesium Form F; and d) isolating the precipitated crystalline pantoprazole magnesium Form F from the mixture.

Form F can be used to make Form A. The invention also encompasses a process for preparing crystalline pantoprazole magnesium Form A comprising: dissolving crystalline pantoprazole Form F in methanol to form a solution; and adding water to the solution to precipitate the crystalline pantoprazole magnesium Form A.

The invention also encompasses process for preparing crystalline pantoprazole magnesium Form A comprising: combining crystalline pantoprazole Form F with a solvent selected from the group consisting of water, ethanol, isopropanol, and mixtures of water and a C₁-C₄ alcohol to obtain a slurry of the crystalline pantoprazole magnesium Form A.

In another embodiment, the invention encompasses a crystalline form of pantoprazole magnesium (designated as Form G) characterized by at least one of: a powder X-ray diffraction pattern having peaks at about 7.2, 12.9, 13.8, and 17.0±0.2° 2θ; a powder X-ray diffraction pattern having peaks at about 7.2, 12.9, 13.8, 14.6, 17.0, 21.7, and 22.7±0.2° 2θ; a powder X-ray diffraction pattern substantially as depicted in FIG. 10; an infrared spectrum having bands at about 3657, 2976, 1647, 1420, 1405, 1179, and 1066±2 cm⁻¹; an IR spectrum substantially as depicted in FIG. 17; a solid state ¹³C NMR spectrum having signals at about 108.2, 113.4, 143.6, 157.1±0.2 ppm; a SSNMR spectrum substantially as depicted in FIG. 21; a solid state ¹³C NMR spectrum having chemical shift differences between the signal exhibiting the lowest chemical shift and another in the chemical shift range of 100 to 200 ppm of about 0, 5.2, 35.4 and 48.9±0.1 ppm. Form G is a hemipentahydrate and contains about 5.1-6.2% of water by weight.

The invention also encompasses a process for preparing the crystalline pantoprazole magnesium Form G comprising exposing crystalline pantoprazole magnesium Form F to about 0-20% relative humidity at about room temperature for example for about 2-7 days.

In another embodiment, the invention encompasses a crystalline form of pantoprazole magnesium (designated as Form H) characterized by at least one of: a powder X-ray diffraction pattern having peaks at about 6.8, 9.0, 9.5, 12.9, and 13.8±0.2° 2θ; a powder X-ray diffraction pattern having peaks at about 6.8, 9.0, 9.5, 12.9, 13.8, 17.2 and 22.6±0.2° 2θ; a powder X-ray diffraction pattern substantially as depicted in FIG. 11; an infrared spectrum having bands at about 3651, 1588, 1424, 1410, and 468±2 cm⁻¹; an IR spectrum substantially as depicted in FIG. 18; a SSNMR spectrum substantially as depicted in FIG. 22, a solid state ¹³C NMR spectrum having signals at about 107.6, 144.1, 157.8, 159.5±0.2 ppm; a solid state ¹³C NMR spectrum has chemical shift differences between the signal exhibiting the lowest chemical shift and another in the chemical shift range of 100 to 200 ppm of about 0, 36.5, 50.2 and 51.9±0.1 ppm. Form H is a tetrahydrate and contains about 8.0-9.0% of water by weight.

The invention also encompasses a process for preparing the crystalline pantoprazole magnesium Form H comprising exposing crystalline pantoprazole magnesium Form F to about 40-100% relative humidity at about room temperature for example for about 4-7 days.

In another embodiment, the invention encompasses a pharmaceutical composition comprising a therapeutically effective amount of at least one of amorphous pantoprazole magnesium, and crystalline pantoprazole magnesium Forms C, E, G, and H and at least one pharmaceutically acceptable excipient.

The invention also encompasses a method of inhibiting gastric acid secretion comprising administering the pharmaceutical composition to a patient in need thereof.

### BRIEF DESCRIPTION OF THE DRAWINGS

FIG. 1 illustrates a powder X-ray diffraction pattern of wet amorphous pantoprazole magnesium prepared according to Example 1.

FIG. 2 illustrates a powder X-ray diffraction pattern of dry amorphous pantoprazole magnesium prepared according to Example 1.

FIG. 3 illustrates a powder X-ray diffraction pattern of amorphous pantoprazole magnesium prepared according to Example 3a.

FIG. 4 illustrates a powder X-ray diffraction pattern of amorphous pantoprazole magnesium prepared according to Example 3b.

FIG. 5 illustrates a powder X-ray diffraction pattern of crystalline pantoprazole magnesium Form C prepared according to Example 4c.

FIG. 6 illustrates a powder X-ray diffraction pattern of crystalline pantoprazole magnesium Form A prepared according to Example 5.

FIG. 7 illustrates a powder X-ray diffraction pattern of crystalline pantoprazole magnesium Form E prepared according to Example 6.

FIG. 8 illustrates a powder X-ray diffraction pattern of crystalline pantoprazole magnesium Form F prepared according to Example 9.

FIG. 9 illustrates a powder X-ray diffraction pattern of crystalline pantoprazole magnesium Form F prepared according to Example 10.

FIG. 10 illustrates a powder X-ray diffraction pattern of crystalline pantoprazole magnesium Form G prepared according to Example 11.

FIG. 11 illustrates a powder X-ray diffraction pattern of crystalline pantoprazole magnesium Form H prepared according to Example 12.

FIG. 12 illustrates an infrared spectrum of amorphous pantoprazole magnesium.

FIG. 13 illustrates an infrared spectrum of crystalline pantoprazole magnesium Form A.

FIG. 14 illustrates an infrared spectrum of crystalline pantoprazole magnesium Form C.

FIG. 15 illustrates an infrared spectrum of crystalline pantoprazole magnesium Form E.

FIG. 16 illustrates an infrared spectrum of crystalline pantoprazole magnesium Form F.

FIG. 17 illustrates an infrared spectrum of crystalline pantoprazole magnesium Form G.

FIG. 18 illustrates an infrared spectrum of crystalline pantoprazole magnesium Form H.

FIG. 19 illustrates a solid state ¹³C nuclear magnetic resonance (“NMR”) spectrum of crystalline pantoprazole magnesium Form E.

FIG. 20 illustrates a solid state ¹³C NMR spectrum of crystalline pantoprazole magnesium Form F.

FIG. 21 illustrates a solid state ¹³C NMR spectrum of crystalline pantoprazole magnesium Form G.

FIG. 22 illustrates a solid state ¹³C NMR spectrum of crystalline pantoprazole magnesium Form H.

FIG. 23 illustrates an analysis of crystalline pantoprazole magnesium Form A by light microscope.

FIG. 24 illustrates an analysis of crystalline pantoprazole magnesium Form E by light microscope.

FIG. 25 illustrates an analysis of crystalline pantoprazole magnesium Form F by light microscope.

FIG. 26 illustrates an analysis of crystalline pantoprazole magnesium Form G by light microscope.

FIG. 27 illustrates an analysis of crystalline pantoprazole magnesium Form H by light microscope.

FIG. 28 illustrates a solid state ¹³C NMR spectrum of crystalline pantoprazole magnesium Form A.

FIG. 29 illustrates a powder X-ray diffraction pattern of crystalline pantoprazole magnesium Form A prepared according to Example 15.

FIG. 30 illustrates a powder X-ray diffraction pattern of crystalline pantoprazole magnesium Form F prepared according to Example 8.

### DETAILED DESCRIPTION OF THE INVENTION

The crystal and amorphous forms of the present invention have properties which makes them suitable for pharmaceutical compositions. These properties, depending on the form, include better morphology, increased solubility and/or more stability, particularly when compared to Form A.

As used herein, the term “PNT” refers to pantoprazole racemate.

As used herein, the term “PNT-Mg” refers to pantoprazole magnesium racemate salt.

As used herein, the term “RH” refers to relative humidity.

As used herein, the term “SSNMR” refers to solid state ¹³C NMR.

The invention encompasses amorphous and crystalline forms of pantoprazole magnesium, as well as processes for their preparation.

As used herein, unless otherwise defined, the term “vacuum” refers to a pressure of less than about 100 mmHg.

As used herein, unless otherwise defines, the term “room temperature” refers to a temperature of about 20° C. to about 30° C., and preferably about 25° C.

The time periods described herein are time periods suitable for laboratory-scale preparations. One of ordinary skill in the art understands that suitable time periods will vary based upon the amounts of reagents present, and can adjust the time periods accordingly.

The present invention provides crystalline forms and amorphous form of pantaprazole magnesium. Also provided are solvated and hydrated crystalline forms, particularly PNT-Mg dimethanolate (corresponds to about 7 wt % methanol content), PNT-Mg hemipentahydrate (corresponds to about 5.1-6.2 wt % water content), and PNT-Mg tetrahydrate (corresponds to about 8.0-9.0 wt % water content).

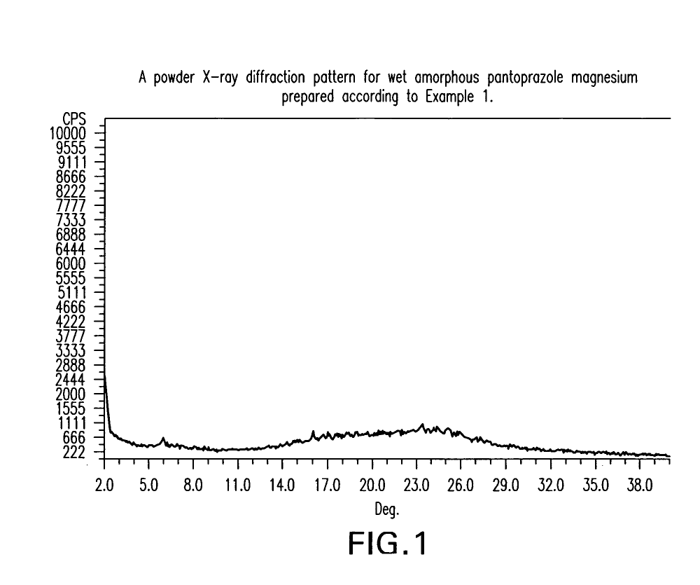

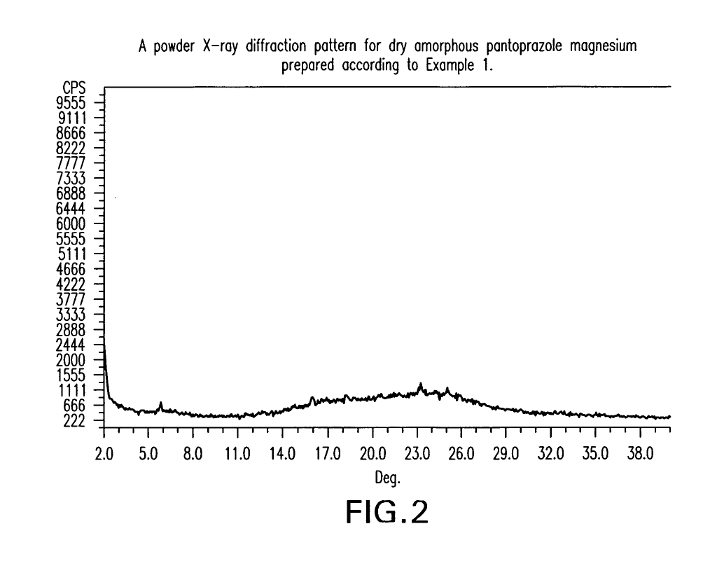

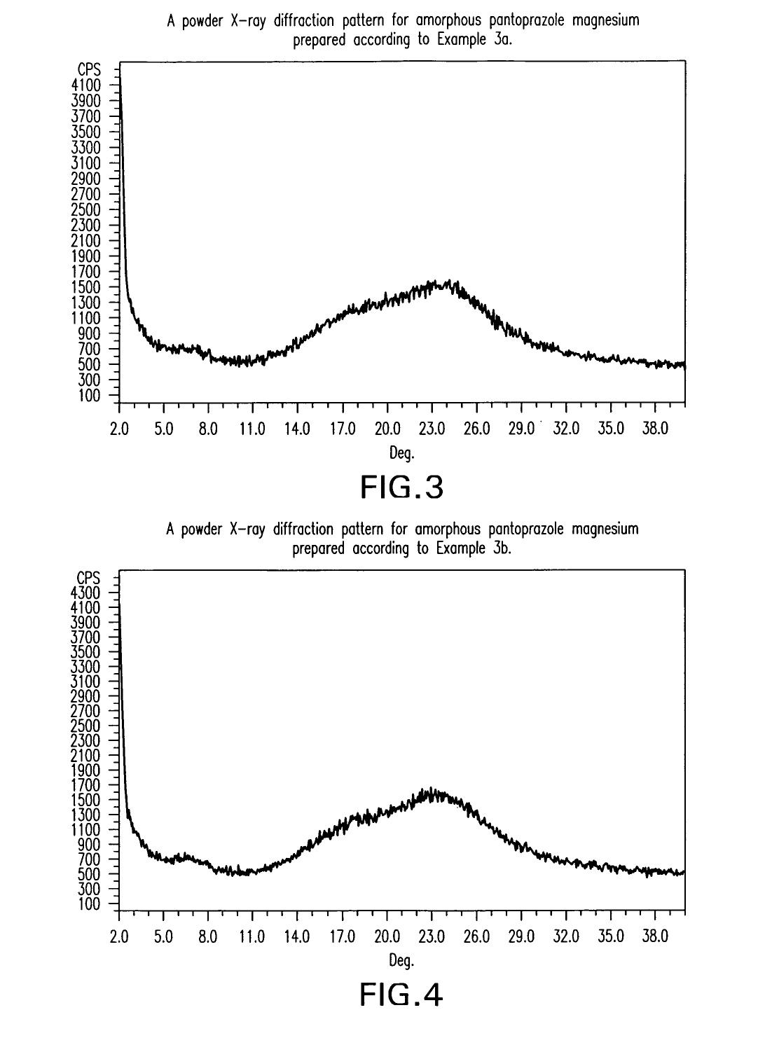

In one embodiment, the invention encompasses amorphous pantoprazole magnesium. The amorphous pantoprazole magnesium may have a PXRD pattern as substantially depicted in FIG. 1, FIG. 2, FIG. 3 or FIG. 4.

The invention also encompasses substantially amorphous pantoprazole magnesium.

Preferably, the amorphous pantroprazole magnesium contains not more than about 10% of crystalline pantroprazole magnesium as measured by area percentage using XRD. The amount of crystalline PNT Mg is quantified by methods known in the art like “crystallinity index” available to most XRD softwares.

In one embodiment, the amorphous pantroprazole magnesium contains not more than about 10% weight percent, more preferably not more than about 5% weight percent and most preferably not more than about 1% weight percent of crystalline pantoprazole magnesium dihydrate Form A or form C as measured by area percentage using XRD. The amount of crystalline pantoprazole magnesium dihydrate Form A or form C present may be determined by powder X-ray diffraction (“PXRD”) based upon at least one of the following characteristic peaks of Form A and form C: 5.8, 14.9, 16.0, 16.6, and 18.3±0.2° 2θ.

The Applicants have found that amorphous pantoprazole magnesium has greater solubility in relation to form A and thus provides greater bioavailability. Specifically, Applicants have compared the solubility of amorphous pantoprazole magnesium to that of crystalline pantoprazole magnesium Form A prepared according to claim 10 of the '464 patent and found that the amorphous form is approximately 6.5 times more soluble in water than crystalline Form A. See Example 16 below.

The invention also encompasses a process for preparing amorphous pantoprazole magnesium comprising dissolving pantoprazole magnesium in a solvent selected from the group consisting of at least one of methanol and ethanol; and removing the solvent to obtain the amorphous pantoprazole magnesium.

The pantoprazole magnesium and solvent may be heated to dissolve the pantoprazole magnesium. Preferably, the pantoprazole magnesium and solvent are heated at a temperature of about 40° C. to about 80° C., and more preferably at about reflux temperature of the solvent, to dissolve the pantoprazole magnesium.

The solvent may be removed by any method known to one of skill in the art. Typically, the solvent is removed by evaporation.

Preferably, the solvent is removed by spray drying. The spray drying technique can easily be applied on an industrial scale and avoids thermal deterioration of the product by the very short contact time between the hot air flow and the amorphous pantoprazole magnesium. Spray-drying involves breaking up liquid mixtures into small droplets (atomization) and rapidly removing solvent from the mixture. The removal of the solvent is preferably accomplished by providing a drying gas. The drying gas may be any suitable gas, although inert gases such as nitrogen, nitrogen-enriched air, and argon are preferred. Preferably, the spray drying apparatus comprises a drying chamber, atomizing means for atomizing a solvent-containing feed into the drying chamber, a source of drying gas that flows into the drying chamber to remove solvent from the atomized-solvent-containing feed, an outlet for the products of drying, and product collection means located downstream from the drying chamber. Examples of such apparatuses include Niro Models PSD-1, PSD-2 and PSD-4 (Niro A/S, Soeborg, Denmark). Preferably, the solution of pantoprazole magnesium is spray-dried using an inlet temperature of about 30° C. to about 150° C., and more preferably about 50° C. to about 130° C. Preferably, the solution of pantoprazole magnesium is spray-dried at an outlet temperature of about 34° C. to about 84° C. The amorphous pantoprazole magnesium produced by spray drying may be recovered by using a cyclone or a filter. Spray drying processes and equipment are described in PERRY'S CHEMICAL ENGINEER'S HANDBOOK, 20-54 to 20-57 (6th ed. 1984), hereby incorporated by reference.

Optionally, the amorphous pantoprazole magnesium thus obtained (wet amorphous pantoprazole magnesium) may be dried. Preferably, the amorphous pantoprazole magnesium is dried under vacuum. More preferably, the amorphous pantoprazole magnesium is dried under vacuum with heating. Preferably, the amorphous pantoprazole magnesium is dried under vacuum at a temperature of about 40° C. to about 85° C., and more preferably at a temperature of about 55° C.

In another embodiment, the invention encompasses a crystalline form of pantoprazole magnesium designated as Form C. Crystalline Form C is more stable to polymorphic transformation under heating than Form A. See Example 17 below. As used herein with relation to form C stable means that the crystal form does not change when heated at 100° C. for two weeks.

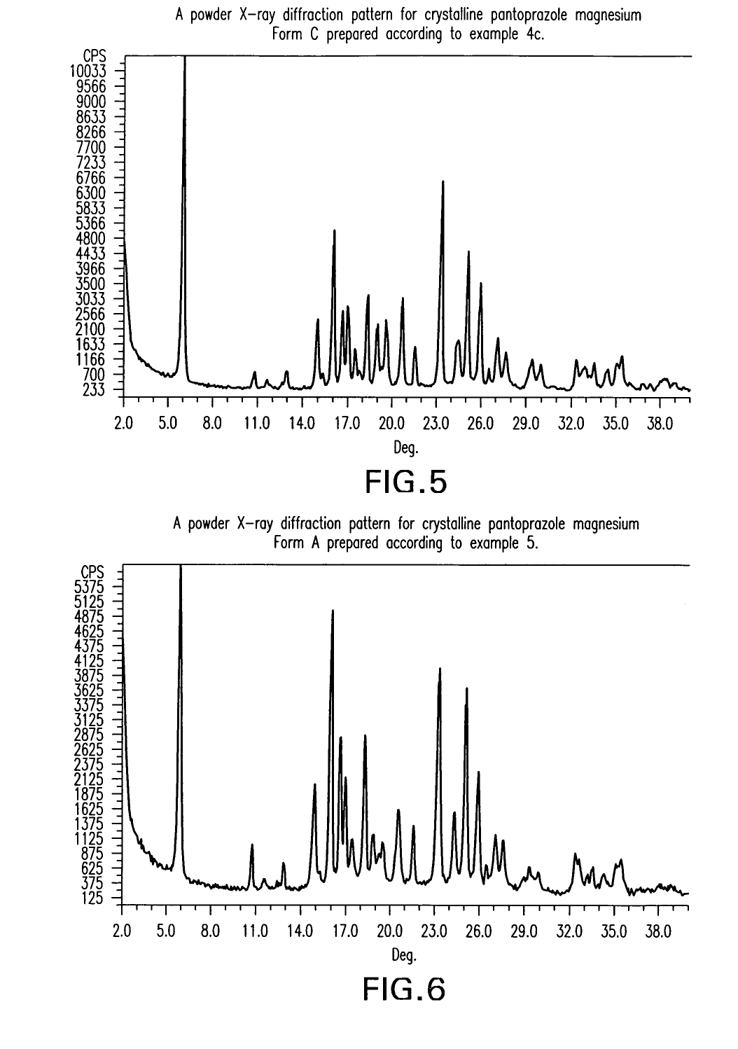

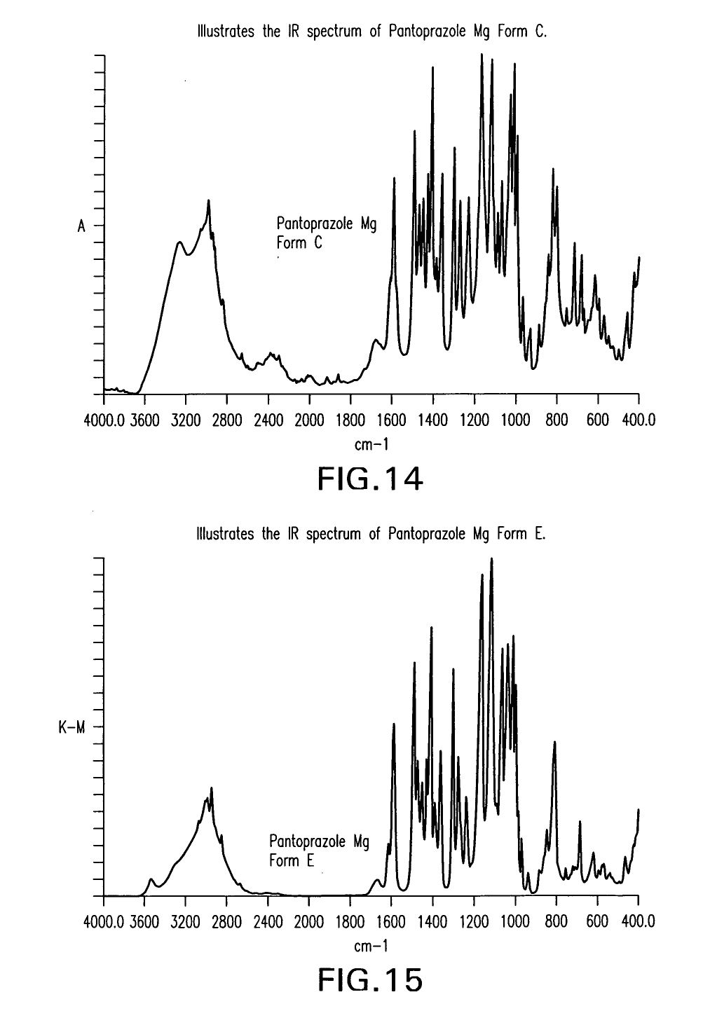

The crystalline pantoprazole magnesium Form C characterized by: at least one of a powder X-ray diffraction (“PXRD”) pattern having peaks at about 6.0, 16.0, 19.0 and 19.6±0.2° 2θ; a PXRD pattern having peaks at about 6.0, 16.0, 19.0, 19.6 and 23.3±0.2° 2θ; a PXRD pattern having peaks at about 16.0, 16.6, 18.3, 19.0 and 19.6±0.2° 2θ; a PXRD pattern substantially as depicted in FIG. 5; a PXRD pattern having peak position differences between the peak at 15.0±0.4° 2θ of about 1.0, 1.6, 3.3, 4.0 and 4.6±0.10° 2θ; an infrared spectrum having bands at about 3275, 2991, 1593, 1428, 1074, 1035 and 827±2 cm⁻¹; an IR spectrum substantially as depicted in FIG. 14. The crystalline form may be further characterized by a melting point of about 185° C.

Crystalline pantoprazole magnesium Form C may be a dihydrate. Preferably, the crystalline pantoprazole magnesium Form C has a water content of about 4.4%-4.7% by weight.

The invention also encompasses a process for preparing the crystalline pantoprazole magnesium Form C comprising combining pantoprazole magnesium Form A with methanol, ethanol, MTBE, ethyl acetate or mixtures thereof to obtain a slurry of crystalline pantoprazole magnesium Form C; heating; and isolating the crystalline pantoprazole magnesium Form C from the slurry. In one embodiment, the solvent is methanol. In another embodiment the solvent is ethanol. In another embodiment the solvent is MTBE. In another embodiment the solvents is ethyl acetate.

Typically, the slurry is heated to obtain the crystalline pantoprazole magnesium Form C. Preferably, the slurry is heated at a temperature of about 40° C. to about 65° C., and more preferably at a temperature of about 65° C. Preferably, the slurry is stirred with heating for a period of time sufficient to obtain the crystalline pantoprazole magnesium Form C. Typically, the slurry is stirred with heating for about 1 to about 24 hours. A slurry is typically obtained at concentrations of less than about 15 volumes.

The crystalline pantoprazole magnesium Form C may be isolated from the slurry by any method known to one of ordinary skill in the art. Preferably, the crystalline pantoprazole magnesium Form C is isolated from the slurry by filtration. Optionally, the isolated crystalline pantoprazole magnesium Form C may be dried. Preferably, the isolated crystalline pantoprazole magnesium Form C is dried at a temperature of about 45° C. to about 70° C., and more preferably at a temperature of about 55° C. Preferably, the isolated crystalline pantoprazole magnesium Form C is dried under vacuum.

In another embodiment, the invention encompasses a crystalline form of pantoprazole magnesium designated as Form E. Crystalline Form E has a smaller particle size than Form A, which eliminates the need for milling before use in a pharmaceutical composition. See Example 18 below.

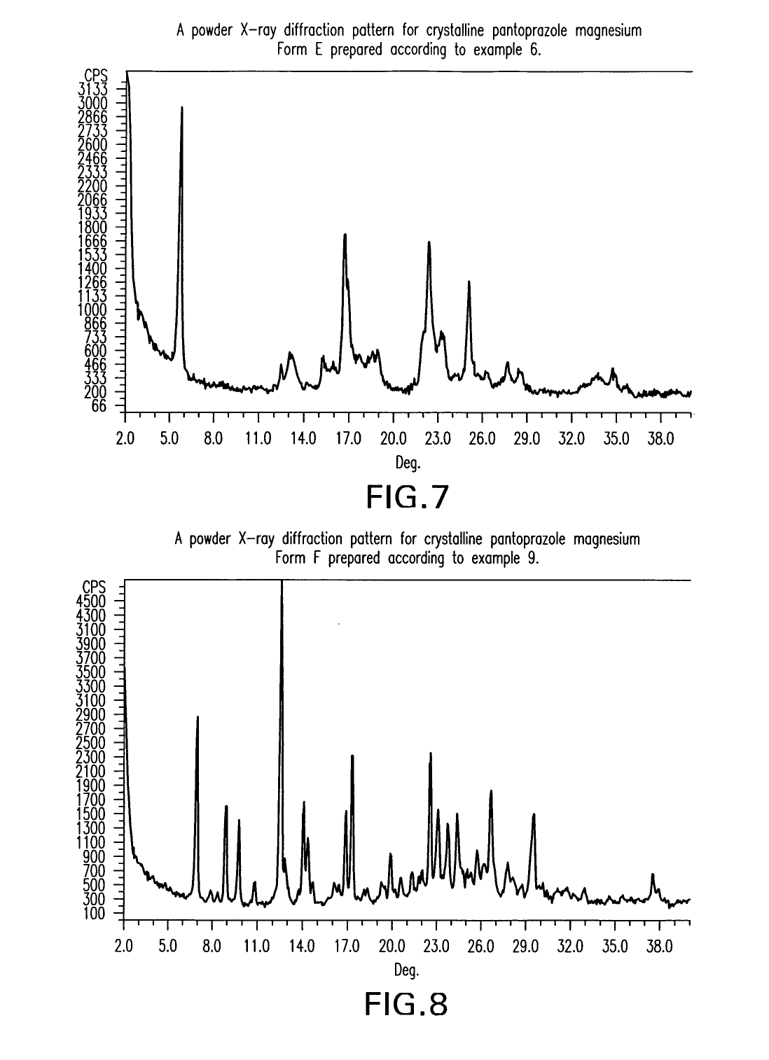

The crystalline pantoprazole magnesium Form E characterized by a PXRD pattern having peaks at about 5.6, 13.1, and 16.6±0.2° 2θ; a PXRD pattern having peaks at about 5.6, 13.1, 16.6, and 22.3±0.2° 2θ; a PXRD pattern having peaks at about 5.6, 12.5, 13.1, and 16.6±0.2° 2θ; a PXRD pattern substantially as depicted in FIG. 7; an infrared spectrum having bands at about 3532, 1664, 1412, 1388, 1002, and 883±2 cm⁻¹; an infrared spectrum substantially as depicted in FIG. 15; a solid state ¹³C NMR spectrum having signals at about 102.5, 118.3, 143.8 and 157.7±0.2 ppm; a the solid state ¹³C NMR spectrum having chemical shift differences between the signal exhibiting the lowest chemical shift and another in the chemical shift range of 100 to 200 ppm of about 0, 15.8, 41.3 and 55.2±0.1 ppm, the signal exhibiting the lowest chemical shift in the chemical shift area of 100 to 200 ppm is typically at about 102.5±1 ppm; a solid state ¹³C NMR spectrum substantially as depicted in FIG. 19. The crystalline form may be further characterized by a melting point of about 175° C.

The crystalline pantoprazole magnesium Form E is a hemipentahydrate. Preferably, the crystalline pantoprazole magnesium Form E has a water content of about 5.1-6.2% by weight.

The invention also encompasses a process for preparing the crystalline pantoprazole magnesium Form E comprising combining pantoprazole free acid, a source of magnesium selected from the group consisting of Mg, Mg(OCH₂CH₃)₂ and Mg(OCH₃)₂, methanol and water to obtain a mixture; heating the mixture; cooling the mixture to precipitate crystalline pantoprazole magnesium Form E; and isolating the precipitated crystalline pantoprazole magnesium Form E from the mixture.

Preferably, the mixture is heated at a temperature of about 40° C. to about reflux temperature, and more preferably at about reflux temperature. Preferably, the mixture is heated for about 1 to about 10 hours, and more preferably for about 2 hours.

Typically, the methanol is removed by evaporation. After the additional amount of methanol is added to the mixture, the mixture is typically stirred. Preferably, the mixture is stirred at a temperature of about 10° C. to about 40° C., and more preferably at about room temperature. Preferably, the mixture is stirred for about 1 hour to about 24 hours, and more preferably for about 20 hours. Preferably after stirring, a further amount of methanol is added.

Preferably, the mixture is cooled at a temperature of about 0° C. to about 10° C., and more preferably at a temperature of about 2° C. Preferably, the mixture is cooled for about 1 hour to about 24 hours, and more preferably for about 2 hours.

The precipitated crystalline pantoprazole magnesium Form E may be isolated from the mixture by any method known to one of ordinary skill in the art. Preferably, the precipitated crystalline pantoprazole magnesium Form E is isolated from the mixture by filtration. Optionally, the isolated crystalline pantoprazole magnesium Form E may be further purified by washing and drying. Preferably, the isolated crystalline pantoprazole magnesium Form E is washed with methanol. Preferably, the isolated crystalline pantoprazole magnesium Form E is dried at a temperature of about 40° C. to about 60° C., and more preferably at a temperature of about 55° C. under vacuum.

In another embodiment, the invention encompasses a crystalline form of pantoprazole magnesium designated as Form F. Crystalline Form F is made up of spherical particles, which are often more flowable than the plate-shaped particles characteristic of Form A. See Example 18 below.

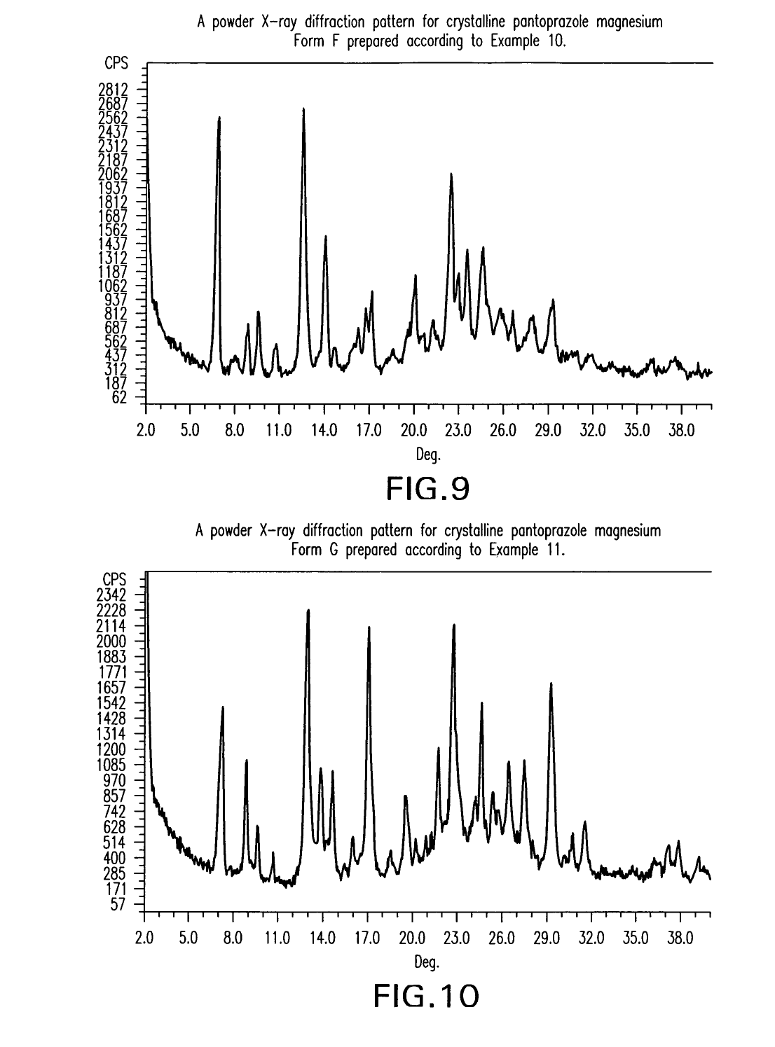

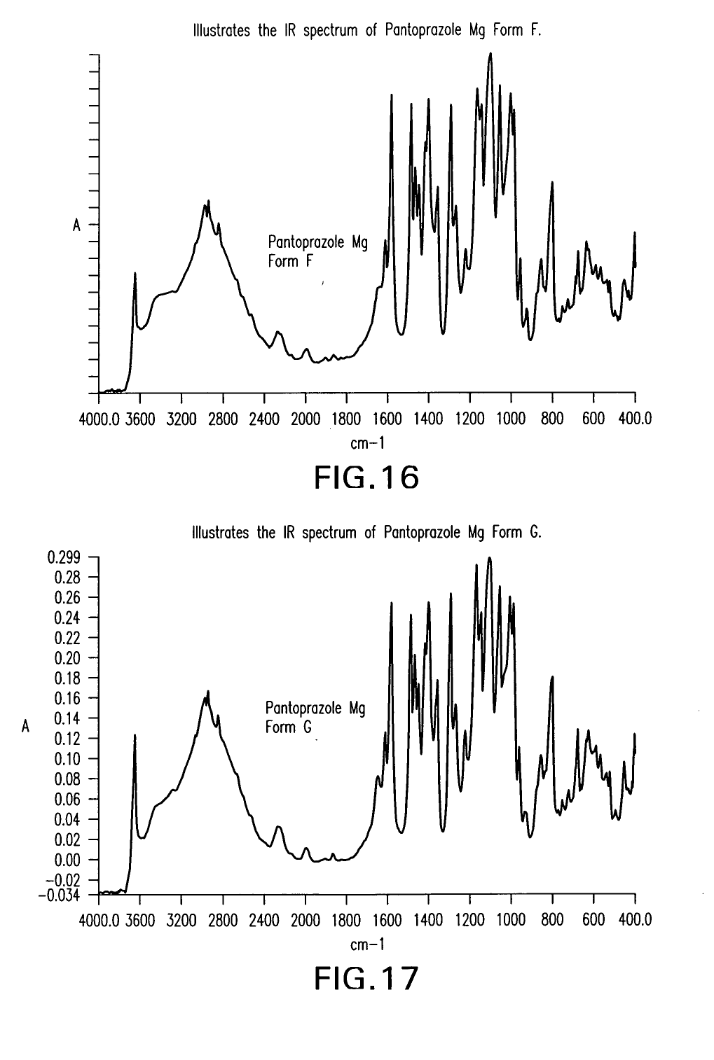

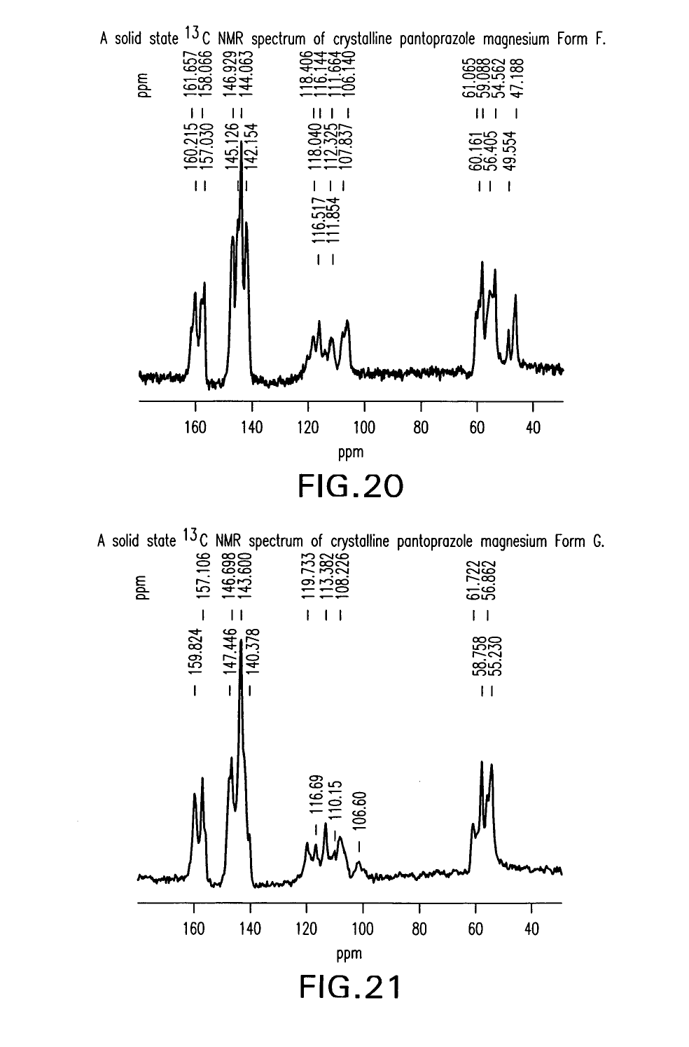

The crystalline pantoprazole magnesium Form F characterized by a PXRD pattern having peaks at about 6.9, 8.9, 9.7, 12.4, and 14.0±0.2° 2θ; a PXRD pattern having peaks at about 6.9, 8.9, 9.7, 12.4, 14.0, 16.9, 17.2, and 22.5±0.2° 2θ.; a PXRD pattern substantially as depicted in FIG. 8 or 9; an infrared spectrum having bands at about 3657, 2982, 1587, 1408, 1176, 1156, and 1069±2 cm⁻¹; an infrared spectrum substantially as depicted in FIG. 16; a solid state ¹³C NMR spectrum having signals at about 106.1, 142.2, 144.0 and 160.2±0.2 ppm; a solid state ¹³C NMR spectrum having chemical shift differences between the signal exhibiting the lowest chemical shift and another in the chemical shift range of 100 to 200 ppm of about 0, 36.1, 37.9 and 54.1±0.1 ppm, the signal exhibiting the lowest chemical shift in the chemical shift area of 100 to 200 ppm is typically at about 106.1±1 ppm; a solid state ¹³C NMR spectrum substantially as depicted in FIG. 20. The crystalline form may be further characterized by a melting point of about 160° C.

The crystalline pantoprazole magnesium Form F may be a dimethanolate and contain about 7 wt % methanol. In addition, the crystalline pantoprazole magnesium Form F may have a water content of about 2.5% by weight.

The invention also encompasses a process for preparing pantoprazole magnesium Form F comprising combining pantoprazole free acid, a source of magnesium selected from the group consisting of Mg, Mg(OCH₃)₂ and Mg(OCH₂CH₃)₂, and methanol to obtain a mixture; heating the mixture; cooling the mixture to precipitate crystalline pantoprazole magnesium Form F; and isolating the precipitated crystalline pantoprazole magnesium Form F from the mixture.

Optionally, the source of magnesium and methanol may be combined and the combination heated prior to adding the pantoprazole free acid. Preferably, when the source of magnesium is Mg₍ₛ₎ methylene chloride is combined with the magnesium and the methanol.

Preferably, the mixture is heated at a temperature of about 40° C. to about reflux temperature, and more preferably at about reflux temperature. Preferably, the mixture is heated for about 1 hour to about 24 hours, and more preferably for about 2 hours to about 10 hours.

Typically, the mixture is stirred prior to the cooling step. Preferably, the mixture is stirred for about 1 hour to about 24 hours, and more preferably for about 4 hours. Preferably, the mixture is stirred at a temperature of about 40° C. to about reflux temperature, and more preferably at about 50° C.

Preferably, the mixture is cooled at a temperature of about 10° C. to about 30° C., and more preferably at about room temperature. Preferably, the mixture is cooled for about 1 hours to about 24 hours, and more preferably for about 2 hours to about 10 hours.

The precipitated crystalline pantoprazole magnesium Form F may be isolated from the mixture by any method known to one of ordinary skill in the art. Preferably, the precipitated crystalline pantoprazole magnesium Form F is isolated from the mixture by filtration. Optionally, the isolated crystalline pantoprazole magnesium Form F may be further purified by washing and drying. Preferably, the isolated crystalline pantoprazole magnesium Form F is washed with methanol. Preferably, the isolated crystalline pantoprazole magnesium Form F is dried at a temperature of about 40° C. to about 60° C., and more preferably at a temperature of about 50° C. under vacuum. At higher temperatures, such as amount 80° C., Form F converts to Form G.

The invention also encompasses a process for preparing the crystalline pantoprazole magnesium Form A comprising combining the crystalline pantoprazole magnesium Form F described below with a solvent selected from the group consisting of water, ethanol, isopropanol (“IPA”), and mixtures of water and a C₁-C₄ alcohol to obtain a slurry of crystalline pantoprazole magnesium Form A.

Preferably, the C₁-C₄ alcohol is methanol.

Typically, the slurry is stirred for a period of time sufficient to obtain the crystalline pantoprazole magnesium Form A. Preferably, the slurry is stirred for about 2 hours to about 24 hours, and more preferably for about 4 hours, to obtain the crystalline pantoprazole magnesium Form A. Preferably, the slurry is stirred at a temperature of about 15° C. to about 40° C., and more preferably at about room temperature.

The crystalline pantoprazole Form A may be isolated from the slurry by any method known to one of ordinary skill in the art. Preferably, the crystalline pantoprazole Form A is isolated from the slurry by filtration.

Optionally, the isolated crystalline pantoprazole magnesium Form A may be dried. Preferably, the isolated crystalline pantoprazole magnesium Form A is dried at a temperature of about 55° C. Preferably, the isolated crystalline pantoprazole magnesium Form A is dried under vacuum.

The invention also encompasses a process for preparing the crystalline pantoprazole magnesium Form A comprising dissolving a crystalline form of pantoprazole magnesium described below in methanol to form a solution and adding water to the solution to precipitate the pantoprazole magnesium Form A.

The invention also encompasses a process for preparing the crystalline pantoprazole magnesium Form A comprising dissolving crystalline pantoprazole magnesium Form F in methanol to form a solution and adding water to the solution to precipitate the pantoprazole magnesium Form A.

Typically, the crystalline pantoprazole Form F and methanol are heated to dissolve the crystalline pantoprazole Form F. Preferably, the crystalline pantoprazole Form F and methanol are heated to about reflux temperature.

Typically, the solution is cooled prior to the addition of water. Preferably, the solution is cooled to about room temperature. Optionally, the cooled solution may be treated with active carbon. Preferably, the treated solution is concentrated by removing the solvent under vacuum at a temperature of about 30° C. to about 60° C., and more preferably at a temperature of about 40° C. Preferably, after the concentration, the obtained mixture is cooled. Preferably, the mixture is cooled to a temperature of about 110° C. to about 30° C., and more preferably to about room temperature.

Preferably, the water is added drop-wise to the solution to obtain a slurry. Preferably, the slurry is stirred to precipitate the crystalline pantoprazole magnesium Form A. Preferably, the slurry is stirred for about 1 hour to about 24 hours, and more preferably for about 2 hours.

The precipitated crystalline pantoprazole Form A may then be recovered from the slurry by any method known to one of ordinary skill in the art. Preferably, the precipitated crystalline pantoprazole Form A is recovered by filtering the precipitated crystalline pantoprazole Form A from the slurry, washing the crystalline pantoprazole Form A, and drying the crystalline pantoprazole Form A. Preferably, the crystalline pantoprazole magnesium Form A is washed with water. Preferably, the crystalline pantoprazole magnesium Form A is dried at a temperature of about 45° C. to about 65° C., and more preferably at a temperature of about 55° C. Preferably, the isolated crystalline pantoprazole magnesium Form A is dried under vacuum.

In another embodiment, the invention encompasses a crystalline form of pantoprazole magnesium designated as Form G. Crystalline Form G has a smaller particle size than Form A, which eliminates the need for milling before use in a pharmaceutical composition. See Example 18 below.

The crystalline pantoprazole magnesium Form G characterized by a PXRD pattern having peaks at about 7.2, 12.9, 13.8, and 17.0±0.2° 2θ; a PXRD pattern having peaks at about 7.2, 12.9, 13.8, 14.6, 17.0, 21.7, and 22.7±0.2° 2θ; a PXRD pattern substantially as depicted in FIG. 10; an infrared spectrum having bands at about 3657, 2976, 1647, 1420, 1405, 1179, and 1066±2 cm⁻¹; an infrared spectrum substantially as depicted in FIG. 17; a solid state ¹³C NMR spectrum having signals at about 108.2, 113.4, 143.6, 157.1±0.2 ppm; a solid state ¹³C NMR spectrum having chemical shift differences between the signal exhibiting the lowest chemical shift and another in the chemical shift range of 100 to 200 ppm of about 0, 5.2, 35.4 and 48.9±0.1 ppm, the signal exhibiting the lowest chemical shift in the chemical shift area of 100 to 200 ppm is typically at about 108.2±1 ppm; a solid state ¹³C NMR spectrum substantially as depicted in FIG. 21. The crystalline form may be further characterized by a melting point of about 155° C.

The crystalline pantoprazole magnesium Form G is a hemipentahydrate. Preferably, the crystalline pantoprazole magnesium Form G has a water content of about 5.1-6.2% by weight.

The invention also encompasses a process for preparing the crystalline pantoprazole magnesium Form G comprising exposing the crystalline pantoprazole magnesium Form F described above to about 0-20% relative humidity at about room temperature for sufficient time, for example about 2-7 days.

In another embodiment, the invention encompasses a crystalline form of pantoprazole magnesium designated as Form H. Crystalline Form H has a smaller particle size than Form A, which eliminates the need for milling before use in a pharmaceutical composition. See Example 18 below.

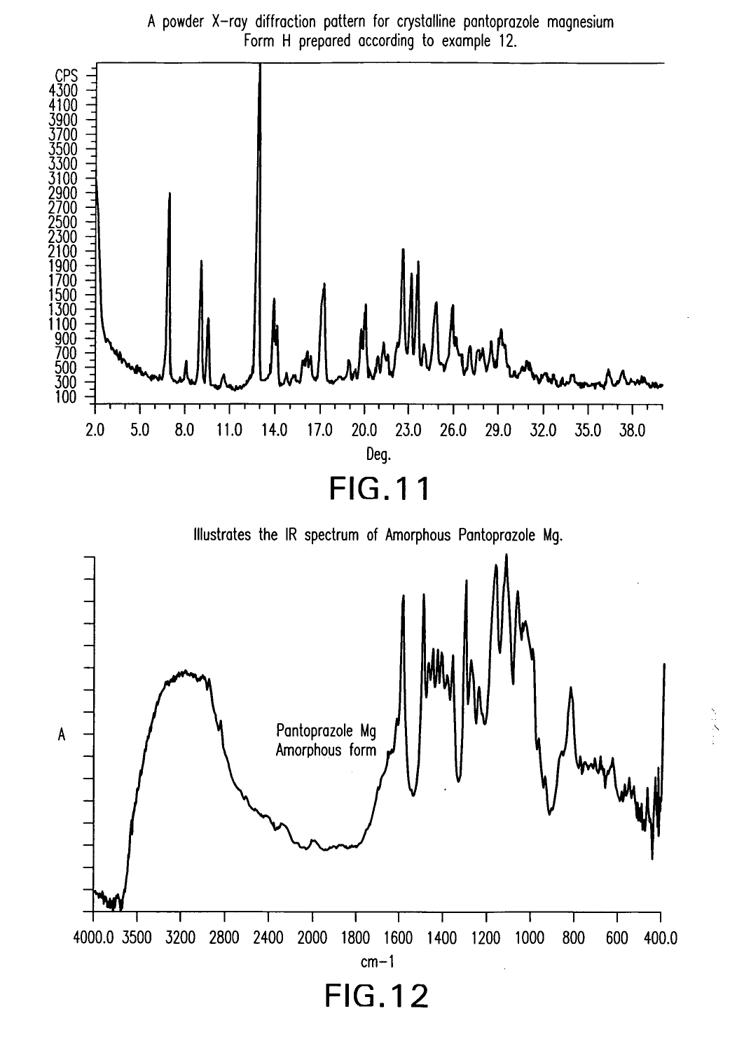

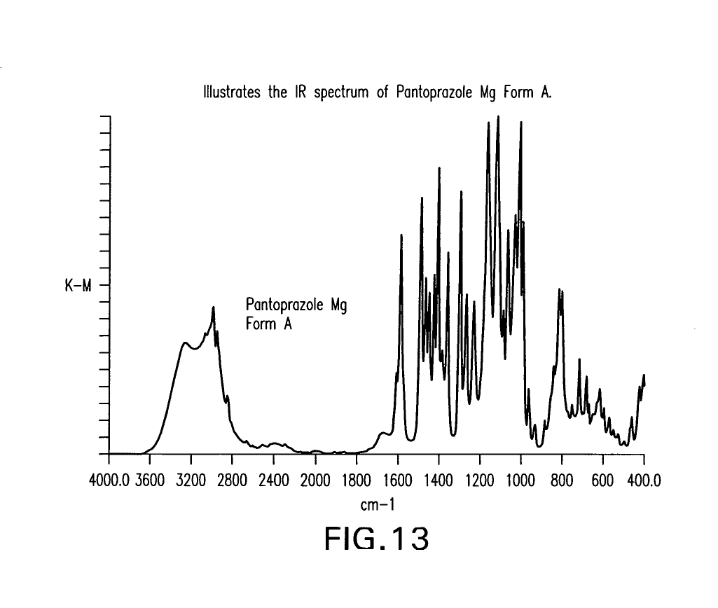

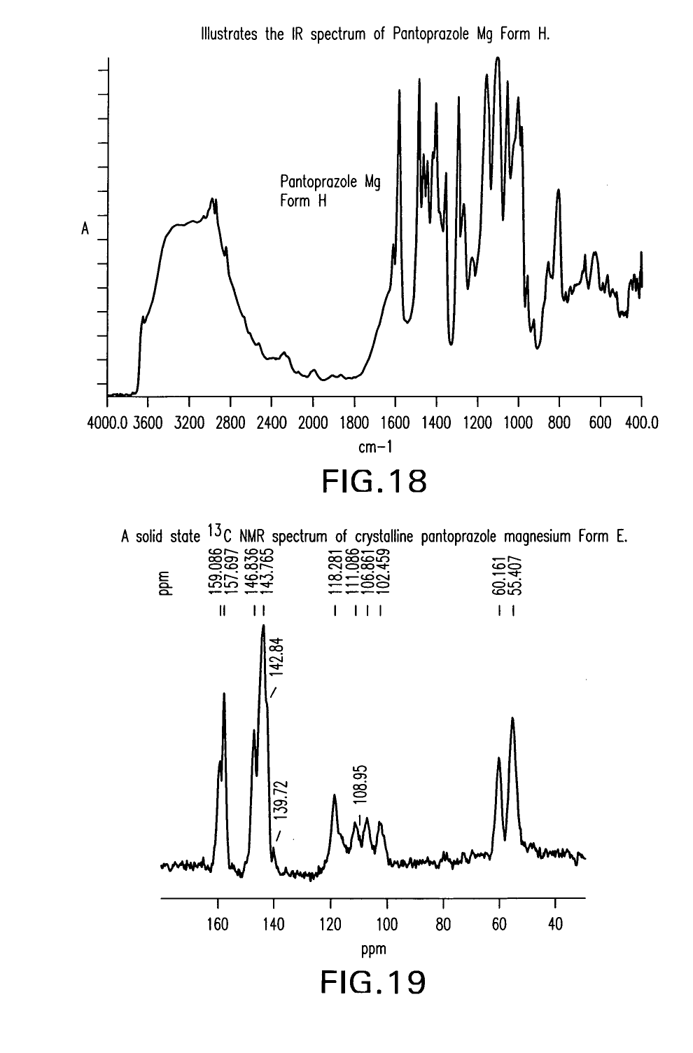

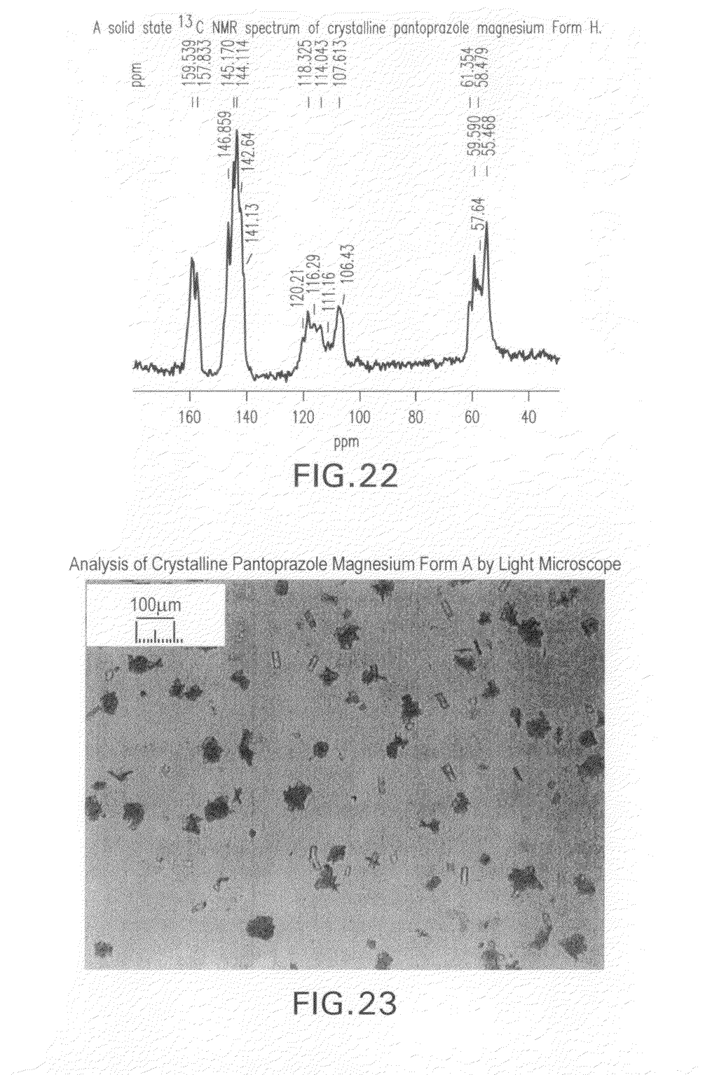

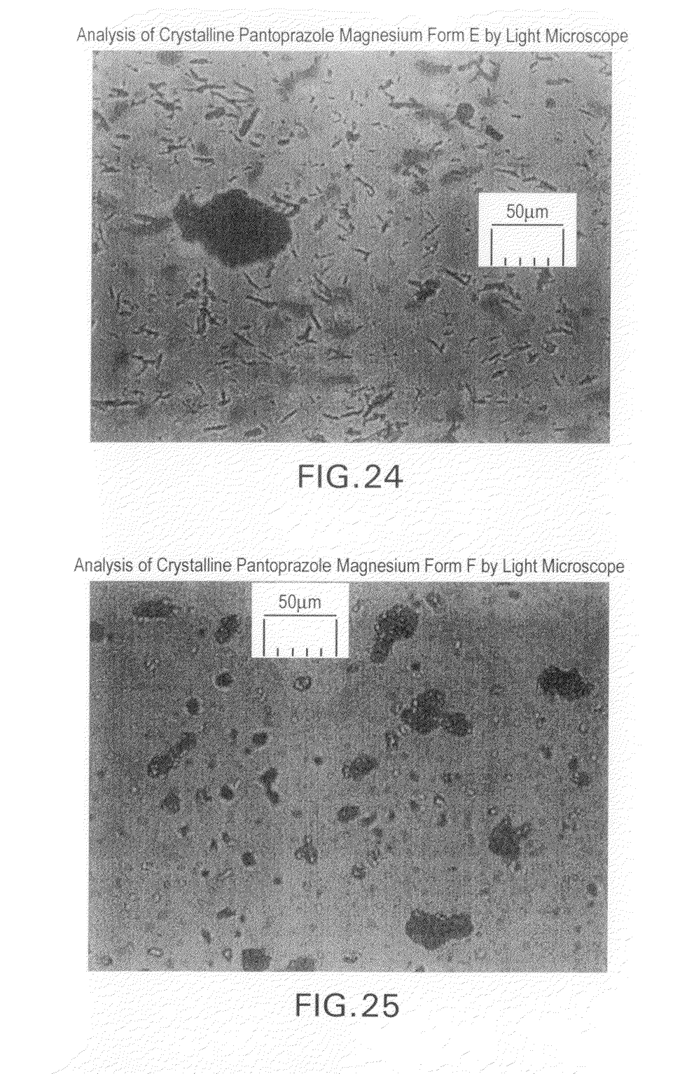

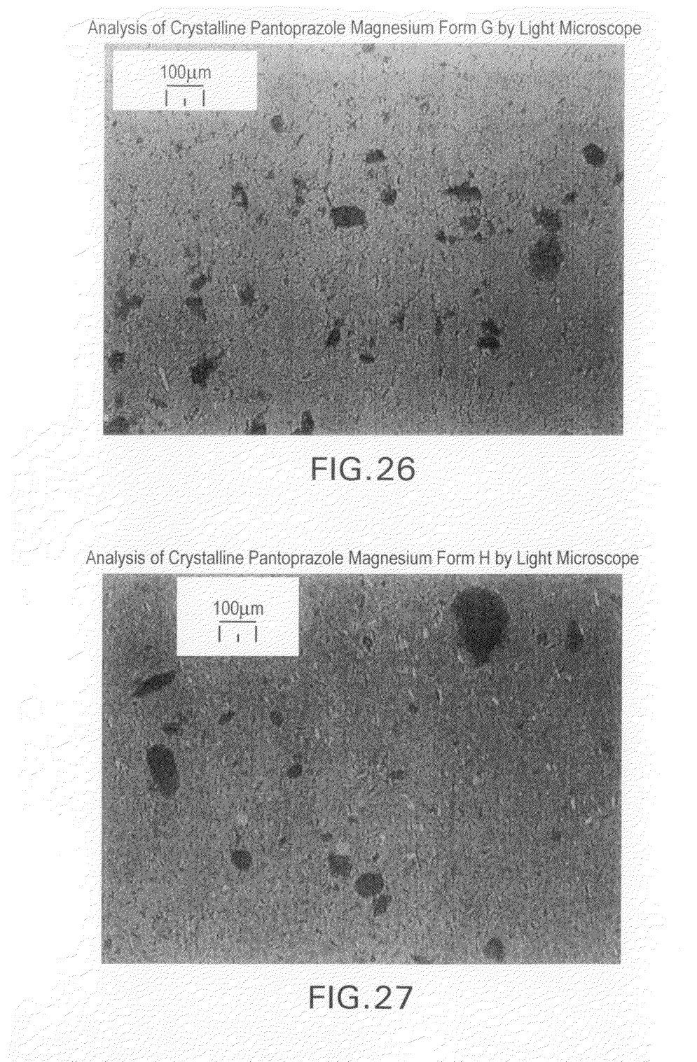

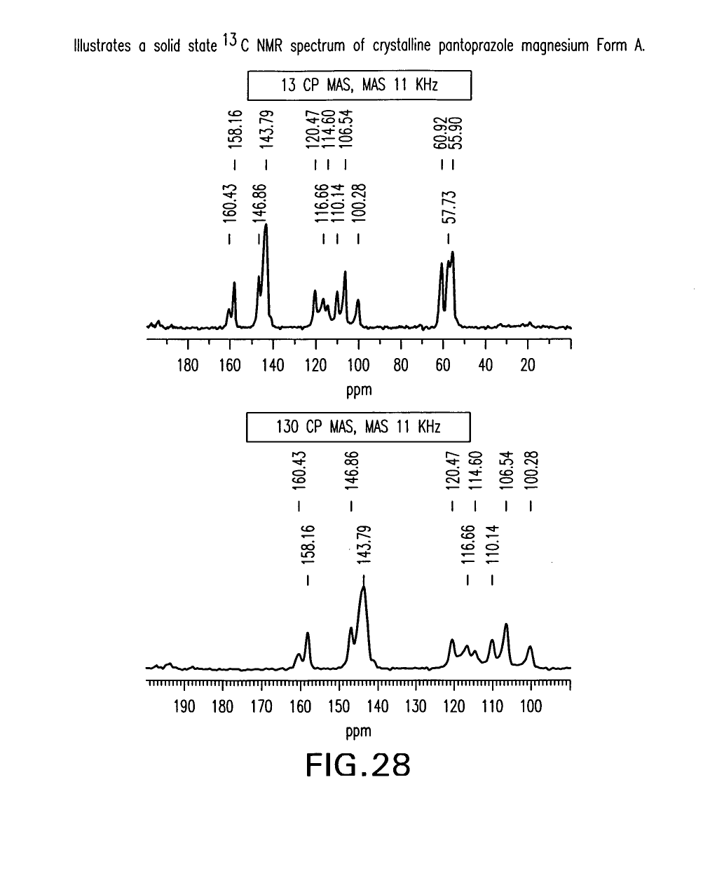

The crystalline pantoprazole Form H is characterized by at least one of: a PXRD pattern having peaks at about 6.8, 9.0, 9.5, 12.9, and 13.8±0.2° 2θ; a PXRD pattern having peaks at about 6.8, 9.0, 9.5, 12.9, 13.8, 17.2 and 22.6±0.2° 2θ; a PXRD pattern substantially as depicted in FIG. 11; an infrared spectrum having bands at about 3651, 1588, 1424, 1410, and 468±2 cm⁻¹; an infrared spectrum substantially as depicted in FIG. 18; a solid state ¹³C NMR spectrum having signals at about 107.6, 144.1, 157.8, 159.5±0.2 ppm; a solid state ¹³C NMR spectrum has chemical shift differences between the signal exhibiting the lowest chemical shift and another in the chemical shift range of 100 to 200 ppm of about 0, 36.5, 50.2 and 51.9±0.1 ppm. The signal exhibiting the lowest chemical shift in the chemical shift area of 100 to 200 ppm is typically at about 107.6±1 ppm; a solid state ¹³C NMR spectrum substantially as depicted in FIG. 22. The crystalline form may be further characterized by a melting point of about 167° C.

The crystalline pantoprazole magnesium Form H may be a tetrahydrate. Preferably, the crystalline pantoprazole magnesium Form H has a water content of about 8.0-9.0% by weight.

The invention also encompasses a process for preparing the crystalline pantoprazole magnesium Form H comprising exposing the crystalline pantoprazole magnesium Form F described above to about 40-100% relative humidity at about room temperature for sufficient time, for example about 2-7 days.

The invention also encompasses pharmaceutical compositions comprising a therapeutically effective amount of at least one of amorphous pantoprazole magnesium, and crystalline pantoprazole magnesium Forms C, E, G, and H and at least one pharmaceutically acceptable excipient.

Formulations can be prepared from amorphous form, Form C, E, G, H as illustrated in example 19.

As used herein, unless otherwise defined, the term “therapeutically effective amount” means an amount sufficient to inhibit gastric acid secretion. The person of ordinary skill in the art would be able to determine a therapeutically effective amount based upon the description herein coupled with the general knowledge in the art. For a particular patient, a therapeutically effective amount may depend on variables such as age, gender, weight, and extent of condition.

The pharmaceutical compositions may be in the form of tablets, pills, powders, liquids, suspensions, emulsions, granules, capsules, suppositories, injection preparations (solutions and suspensions), and the like.

The amount of pantoprazole magnesium contained in the pharmaceutical compositions is not specifically restricted; however, the dose should be sufficient to treat, ameliorate, or reduce the condition to be treated.

Pharmaceutically acceptable excipients may be selected from the group consisting of at least one of diluents, carriers, fillers, bulking agents, binders, disintegrants, disintegration inhibitors, absorption accelerators, wetting agents, lubricants, glidants, surface active agents, flavoring agents, and the like.

Diluents increase the bulk of a solid pharmaceutical composition and can make a pharmaceutical dosage form containing the composition easier for the patient and care giver to handle. Diluents for solid compositions include, but are not limited to, microcrystalline cellulose (e.g., AVICEL®), microfine cellulose, lactose, starch, pregelitinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, or talc.

Carriers for use in the pharmaceutical compositions may include, but are not limited to, lactose, white sugar, sodium chloride, glucose, urea, starch, calcium carbonate, kaolin, crystalline cellulose, or silicic acid.

Binders help bind the active ingredient and other excipients together after compression. Binders for solid pharmaceutical compositions may include, but are not limited to, acacia, alginic acid, carbomer (e.g. CARBOPOL®), carboxymethylcellulose sodium, dextrin, ethyl cellulose, gelatin, guar gum, hydrogenated vegetable oil, hydroxyethyl cellulose, hydroxypropyl cellulose (e.g. KLUCEL®), hydroxypropyl methyl cellulose (e.g. METHOCEL®), liquid glucose, magnesium aluminum silicate, maltodextrin, methylcellulose, polymethacrylates, povidone (e.g. KOLLIDON®, PLASDONE®), pregelatinized starch, sodium alginate, or starch.

Disintegrants can increase dissolution. Disintegrants may include, but are not limited to, alginic acid, carboxymethylcellulose calcium, carboxymethylcellulose sodium (e.g. AC-DI-SOL®, PRIMELLOSE®), colloidal silicon dioxide, croscarmellose sodium, crospovidone (e.g. KOLLIDON®, POLYPLASDONE®), guar gum, magnesium aluminum silicate, methyl cellulose, microcrystalline cellulose, polacrilin potassium, powdered cellulose, pregelatinized starch, sodium alginate, sodium starch glycolate (e.g. EXPLOTAB®) and starch.

Disintegration inhibitors may include, but are not limited to, white sugar, stearin, coconut butter, hydrogenated oils, and the like.

Absorption accelerators may include, but are not limited to, quaternary ammonium base, sodium laurylsulfate, and the like.

Wetting agents may include, but are not limited to, glycerin, starch, and the like. Adsorbing agents used include, but are not limited to, starch, lactose, kaolin, bentonite, colloidal silicic acid, and the like.

Lubricants can be added to the composition to reduce adhesion and ease release of the product from a punch or dye during tableting. Lubricants may include, but are not limited to, magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc and zinc stearate.

Glidants can be added to improve the flowability of non-compacted solid composition and improve the accuracy of dosing. Excipients that can function as glidants may include, but are not limited to, colloidal silicon dioxide, magnesium trisilicate, powdered cellulose, starch, talc, and tribasic calcium phosphate.

Flavoring agents and flavor enhancers make the dosage form more palatable to the patient. Common flavoring agents and flavor enhancers for pharmaceutical products include, but are not limited to, maltol, vanillin, ethyl vanillin, menthol, citric acid, fumaric acid, ethyl maltol, and tartaric acid.

Tablets can be further coated with commonly known coating materials such as sugar coated tablets, gelatin film coated tablets, tablets coated with enteric coatings, tablets coated with films, double layered tablets, and multi-layered tablets. Capsules can be coated with shell made, for example, from gelatin and optionally contain a plasticizer such as glycerin and sorbitol, and an opacifying agent or colorant.

Solid and liquid compositions can also be dyed using any pharmaceutically acceptable colorant to improve their appearance and/or facilitate patient identification of the product and unit dosage level.

In liquid pharmaceutical compositions of the present invention, the pantoprazole magnesium and any other solid ingredients are dissolved or suspended in a liquid carrier, such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, but are not limited to, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain viscosity enhancing agents to improve the mouth-feel of the product and/or coat the lining of the gastrointestinal tract. Such agents may include, but are not limited to, acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as sorbitol, saccharin, sodium saccharin, sucrose, aspartame, fructose, mannitol and invert sugar can be added to improve the taste. Preservatives and chelating agents such as alcohol, sodium benzoate, butylated hydroxy toluene, butylated hydroxyanisole and ethylenediamine tetraacetic acid can be added at safe levels to improve storage stability.

A liquid composition according to the present invention can also contain a buffer such as gluconic acid, lactic acid, citric acid or acetic acid, sodium gluconate, sodium lactate, sodium citrate, or sodium acetate.

Selection of excipients and the amounts to use can be readily determined by an experienced formulation scientist in view of standard procedures and reference works known in the art.

A composition for tableting or capsule filing can be prepared by wet granulation. In wet granulation some or all of the active ingredients and excipients in powder form are blended and then further mixed in the presence of a liquid, typically water, which causes the powders to clump up into granules. The granulate is screened and/or milled, dried and then screened and/or milled to the desired particle size. The granulate can then be tabletted or other excipients can be added prior to tableting, such as a glidant and/or a lubricant.

A tableting composition can be prepared conventionally by dry blending. For instance, the blended composition of the actives and excipients can be compacted into a slug or a sheet and then comminuted into compacted granules. The compacted granules can be compressed subsequently into a tablet.

As an alternative to dry granulation, a blended composition can be compressed directly into a compacted dosage form using direct compression techniques. Direct compression produces a more uniform tablet without granules. Excipients that are particularly well-suited to direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention can comprise any of the aforementioned blends and granulates that were described with reference to tableting, only they are not subjected to a final tableting step.

When shaping the pharmaceutical composition into pill form, any commonly known excipient used in the art can be used. For example, carriers include, but are not limited to, lactose, starch, coconut butter, hardened vegetable oils, kaolin, talc, and the like. Binders used may include, but are not limited to, gum arabic powder, tragacanth gum powder, gelatin, ethanol, and the like. Disintegrating agents used include, but are not limited to, agar, laminalia, and the like.

For the purpose of shaping the pharmaceutical composition in the form of suppositories, any commonly known excipient used in the art can be used. For example, excipients include, but are not limited to, polyethylene glycols, coconut butter, higher alcohols, esters of higher alcohols, gelatin, semisynthesized glycerides, and the like.

When preparing injectable pharmaceutical compositions, solutions and suspensions are sterilized and are preferably made isotonic to blood. Injection preparations may use carriers commonly known in the art. For example, carriers for injectable preparations include, but are not limited to, water, ethyl alcohol, propylene glycol, ethoxylated isostearyl alcohol, polyoxylated isostearyl alcohol, and fatty acid esters of polyoxyethylene sorbitan. One of ordinary skill in the art can easily determine with little or no experimentation the amount of sodium chloride, glucose, or glycerin necessary to make the injectable preparation isotonic. Additional ingredients, such as dissolving agents, buffer agents, and analgesic agents may be added. If necessary, coloring agents, preservatives, perfumes, seasoning agents, sweetening agents, and other medicines may also be added to the desired preparations.

The invention also encompasses processes for preparing the pharmaceutical compositions comprising combining a therapeutically effective amount of at least one of amorphous pantoprazole magnesium, and crystalline pantoprazole magnesium Forms C, E, G, and H and at least one pharmaceutically acceptable excipient.

The invention also encompasses a method of inhibiting gastric acid secretion comprising administering a pharmaceutical composition comprising a therapeutically effective amount of at least one of amorphous pantoprazole magnesium, and crystalline pantoprazole magnesium Forms C, E, G, and H and at least one pharmaceutically acceptable excipient to a patient in need thereof.

The invention also encompasses the use of at least one of amorphous pantoprazole magnesium, and crystalline pantoprazole magnesium Forms C, E, G, and H in the manufacture of a pharmaceutical composition for inhibiting gastric acid secretion.

One of ordinary skill in the art is aware that there is a certain amount of experimental error inherent in powder X-ray diffraction techniques. See, e.g., U.S. PHARMACOPEIA, 387-89 (30th ed. 2007), hereby incorporated by reference. As to individual peaks, peak positions are reported over a range of ±0.2° 2θ to account for this experimental error. As to PXRD patterns in their entirety, the term “substantially as depicted” in a particular figure is meant to account for this experimental error. A PXRD pattern “substantially as depicted” in a particular figure means that one of ordinary skill in the art, understanding the experimental error involved in powder X-ray diffraction techniques, would determine that the PXRD pattern corresponds to the same crystalline structure as the PXRD pattern depicted in the figure.

Having described the invention with reference to certain preferred embodiments, other embodiments will become apparent to one skilled in the art from consideration of the specification. The invention is further defined by reference to the following examples describing in detail the process and compositions of the invention. It will be apparent to those skilled in the art that many modifications, both to materials and methods, may be practiced without departing from the scope of the invention.

### EXAMPLES

### Powder X-Ray Diffraction

The powder X-ray diffraction was performed on Scintag X-ray powder diffractometer model X'TRA with a solid state detector. Copper radiation of 1.5418 Å was used. The sample holder was a round standard aluminum sample holder with rough zero background. The scanning parameters were range: 2-40 degrees two-theta; scan mode: continuous scan; step size: 0.05 deg.; and at a rate of 5 deg/min.

### Differential Scanning Calorimetry

DSC analysis was done using a Mettler 821 Star©. The weight of the samples was about 5 mg; the samples were scanned at a rate of 10° C./min from 25° C. to 200 or 250° C. The oven was constantly purged with nitrogen gas at a flow rate of 40 ml/min. Standard 70 μl alumina crucibles covered by lids with 1 hole were used.

### Infrared Spectroscopy

IR analysis was done using a Perkin Elmer SPECTRUM ONE FT-IR spectrometer in DRIFT mode. The samples in the 4000-400 cm⁻¹ interval were scanned 16 times with 4.0 cm⁻¹ resolution.

### Melting Point

Melting point was determined in BUCHI Melting Point B-545 instrument in capillary.

### Water Content

Water content was determined by Karl Fisher analysis.

### ¹³C NMR Spectroscopy

Pantoprazole Mg Form A was analyzed as follows:

### Experimental:

¹³C CP/MAS NMR spektra were measured using Bruker AVANCE 500 (Karlsruhe, SRN, 2003) spectrometer, frequency 125 and 500 MHz (for ¹³C resp. ¹H). Magic angle spinning (MAS) 11 kHz. Glycine was used to calibrate chemical shifts (glycine shift CO is 176.03 ppm).

Pantoprazole Mg Form E, F, G, H were analyzed as follows:
The cp/mas ¹³C NMR investigations were made at 125.76 MHz and were performed at ambient temperature on a Bruker DMX-500 digital FT NMR spectrometer equipped with a BL-4 cp/mas probehead and High Resolution/High Performance (HPHP) 1H and X-channel pramplifiers for solids. The spectrometer and cp/mas unit were completely calibrated prior to the actual studies.

### Example 1

### Preparation of Amorphous Pantoprazole Magnesium

A 0.1 L flask was loaded with methanol (20 ml) and PNT-Mg Form A (5 g). The resulting mixture was heated until the PNT-Mg dissolved. The solvent was then evaporated to dryness. A sample of the resulting wet solid was taken, analyzed by PXRD, and identified as amorphous form. The solid was then dried at 55° C. under vacuum, analyzed by PXRD and identified as amorphous form.

### Example 2

### Preparation of Amorphous Pantoprazole Magnesium

A 0.1 L flask was loaded with ethanol (150 ml), and PNT-Mg Form A (3 g). The resulting mixture was heated to reflux and the remaining solids were filtered. The solvent was evaporated to dryness. A sample of the resulting wet solid was taken and found to be amorphous form. The solid was then dried at 55° C. under vacuum and found to be amorphous form by PXRD.

### Example 3

### Preparation of Amorphous Pantoprazole Magnesium

PNT-Mg Form A (10 g) was dissolved in methanol (120 ml) at about 65° C. and the solution split into two portions.

a) The first portion of the solution was pumped into a spray dryer at room temperature with nitrogen at an inlet temperature of 50° C. The evaporated solvent and nitrogen exited the spray dryer at 37° C.-38° C. The remaining solid was amorphous PNT-Mg.

b) The second portion of the solution was pumped into a spray dryer at room temperature with nitrogen at an inlet temperature of 130° C. The evaporated solvent and nitrogen exited the spray dryer at 82° C.-84° C. The remaining solid was amorphous PNT-Mg.

### Example 4

### Preparation of Crystalline Pantoprazole Magnesium Forms C

A flask (50 mL) was loaded with a solvent selected from the group consisting of ethyl acetate, methyl tert-butyl ether, ethanol, and methanol, and PNT-Mg Form A (2 g) to form a mixture. The mixture was then heated while stirring to obtain a precipitate of crystalline PNT-Mg. The precipitate of crystalline PNT-Mg was separated from the mixture by filtration to obtain a wet solid, which was analyzed by PXRD. The wet solid was then dried at 55° C. under vacuum and the resulting dry solid was also analyzed by PXRD. The results are summarized in Table 1 below.

| | | Solvent amount | | | | |
|---|---|---|---|---|---|---|
| | | (volumes | | | | |
| | | relative to grams | Heating | | | |
| | | of starting PNT- | temperature | | | Crystal |
| Example | Solvent | Mg) | (° C.) | Time (h) | Sample | form |
| 4a | Ethyl | 5 | 60 | 24 | dry | Form C |
| | acetate | | | | | |
| 4b | Ethanol | 10 | 78 | 1 | dry | Form C |
| 4c | Methanol | 10 | 65 | 1 | wet | Form C |
| | | | | | dry | Form C |

### Example 5

### Preparation of Crystalline Pantoprazole Magnesium Form A

A round bottom flask (3 L) was loaded with MeOH (1.62 L), aqueous ammonia (25%, 105 ml) and pantoprazole free acid (300 g) to form a mixture. The mixture was stirred at room temperature to dissolve the solids. MgSO₄×7H₂O (154 g) was added to the solution and the resulting mixture was stirred for 3 hours at room temperature. The methanol was then evaporated under vacuum and water (3 L) was added to the residue to form a solution. The solution was stirred at 40° C. for 1 hour, during which time a precipitate formed. The precipitated solid was separated by filtration, washed with water (300 ml), and dried at 55° C. under vacuum.

The dry solid (305 g) was added into ethyl acetate (“EtOAc”) (2.25 L) and the mixture was stirred for 1 hour at reflux. The solid was filtered, washed with EtOAc (300 ml) and dried at 55° C. under vacuum. The sample was analyzed by PXRD and found to be pantoprazole Mg Form A.

### Example 6

### Preparation of Crystalline Pantoprazole Magnesium Form E

A flask (250 ml) was loaded with MeOH (50 ml), Mg(OMe)₂ (4.8% in MeOH solution—11.7 g) and pantoprazole free acid (5 g). The mixture was heated to reflux for 2 hours. The solvent was evaporated to dryness. MeOH was added (10 ml), the mixture was stirred for 20 hours at room temperature, and MeOH (15 ml) was added. The mixture was cooled to 2° C. for 2 hours. The solid was filtered, washed with MeOH (10 ml) and dried at 50° C. under vacuum. (63% yield, Chemical Purity 100%).

### Example 7

### Preparation of Crystalline Pantoprazole Magnesium Form E

A flask (1 L) was loaded with methanol (225 ml), Mg(OEt)₂ (12.8 g), water (1.5 g), and pantoprazole free acid (75 g). The mixture was heated to reflux and stirred for 4 h. The mixture was then cooled to 2° C. overnight. The resulting solid was filtered, washed with methanol, and dried at 50° C. under vacuum to give crystalline pantoprazole magnesium Form E. (53% yield).

### Example 8

### Preparation of Crystalline Pantoprazole Magnesium Form F

A flask (250 ml) was loaded with MeOH (33 ml), magnesium (0.42 g), and methylene chloride (0.5 ml). The mixture was heated to reflux for 1 hour. Pantoprazole free acid (11 g) was added, the mixture was stirred 4 hours at 50° C. The mixture was cooled to room temperature for 1 hour. The solid was filtered, washed with MeOH (60 ml) and dried at 50° C. under vacuum. (95% yield, chemical purity 99.94%)

### Example 9

### Preparation of Crystalline Pantoprazole Form F

A flask (250 ml) was loaded with MeOH (75 ml), Mg(OEt)₂ (4.29 g) and pantoprazole free acid (25 g). The mixture was stirred for 4 h at reflux. Then was cooled to room temperature for 1 hour. The solid was filtered, washed with MeOH (50 ml) and dried at 50° C. under vacuum. (93% yield, chemical purity 99.89%)

### Example 10

### Preparation of Crystalline Pantoprazole Form F

A flask (1 L) was loaded with MeOH (330 ml) and magnesium (4.2 g). The mixture was heated to reflux for 1 h. Pantoprazole free acid (110 g) was added at 60° C. The mixture was stirred 4 hours at reflux. The mixture was cooled to room temperature for 2 h. The solid was filtered, washed with MeOH (150 ml) and dried at 50° C. under vacuum. (100% yield, chemical purity 99.79%).

### Example 11

### Preparation of Crystalline Pantoprazole Magnesium Form G

200 mg of crystalline pantoprazole magnesium Form F was placed into a container and stored for 7 days under 0 and 20% relative humidity (“RH”) at room temperature. After storage, the sample was analyzed by PXRD and found to be Form G.

### Example 12

### Preparation of Crystalline Pantoprazole Magnesium Form H

200 mg of crystalline pantoprazole magnesium Form F was placed into a container and stored for 7 days under 40, 60, 80, and 100% RH at room temperature. After storage, the sample was analyzed by PXRD and found to be Form H.

### Example 13

### Preparation of Crystalline Pantoprazole Magnesium Form A

A flask (1 L) was loaded with MeOH (0.6 L) and crystalline pantoprazole magnesium Form F (15 g). The mixture was heated to reflux until dissolution. The solution was cooled to room temperature and active carbon was added (0.75 g, 5%), the mixture was stirred for 1 h at room temperature and then filtered. The solution was concentrated to 20 vol (under vacuum at 40° C.) and then cooled to room temperature. Water was added (90 ml) dropwise over a period of 15 min. The obtained slurry was stirred for 2 h at room temperature. The precipitate was filtered, washed with water (15 ml) and dried at 55° C. under vacuum. 12.2 g of Pantoprazole-magnesium Form A dry were obtained. (Total yield 86%, Mg-3.1%).

### Example 14

### Preparation of Crystalline Pantoprazole Magnesium Form A

A flask (50 mL) was loaded with solvent (5 vol), and crystalline pantoprazole magnesium Form F (3 g). The mixture was stirred for 4 hours at room temperature. The solid was filtered and dried at 55° C. under vacuum.

| | Solvent amount | |
|---|---|---|
| Solvent type | (volumes) | Crystalline Form |
| Water | 5 | Form A |
| Ethanol | 5 | Form A |
| IPA | 5 | Form A |
| Water:methanol | 2.5:2.5 | Form A |

### Comparative Example 15

### Preparation of 5-Difluoromethoxy-2-[[(3,4-dimethyl-2-pyridinyl)methyl]sulfinyl]-1H-bezimidazole, magnesium salt by repetition of Example 10 of the '464 patent

5-Difluoromethoxy-2-[[(3,4-dimethyl-2-pyridiniyl)methyl]sulfinyl]-1H-bezimidazole (11.1 g, 29 mmol) together with aqueous ammonia (3.8 ml of 25%, 50 mmol) was added to methanol (60 ml). To the solution MgSO₄×7H₂O (5.7 g, 23 mmol) was added. After stirring for 3 minutes the mixture was filtered. Water (40 ml) was added dropwise to the filtrate while stirring. After 30 minutes the product was isolated by filtration and the crystals were washed with methanol/water (25 ml). The product was dried under reduced pressure.

### Example 16

### Comparison of the Solubility of Amorphous Pantoprazole Magnesium and Crystalline Pantoprazole Magnesium Form A

The solubilities of amorphous pantoprazole magnesium and pantoprazole magnesium Form A were each measured in water at 25° C. The certain amount of amorphous sample and form A sample were added each to 50 ml of distillated water. The solutions were kept saturated during the experiment. The aliquots were taken, filtered and injected into HPLC by the method of assay determination for PNT-Mg and the concentrations were calculated by comparison of absorptions at certain wavelength against the known concentration of PNT-Mg standard. Amorphous pantoprazole magnesium was found to have a solubility of about 1.3 mg/ml, while pantoprazole magnesium Form A was found to have a solubility of about 0.2 mg/ml.

### Example 17

### Stability of Crystalline Pantoprazole Magnesium Forms C and A Under Heating

Samples of crystalline pantoprazole magnesium Form C and Form A (prepared according to example 15) were each heated at 100° C. for two weeks. The samples were then analyzed by PXRD. The Form C sample was found to have maintained its crystalline form, while the Form A sample transformed to amorphous form.

### Example 18

### Analysis of Particle Size of Crystalline Pantoprazole Magnesium Forms A, E, F, G, and H

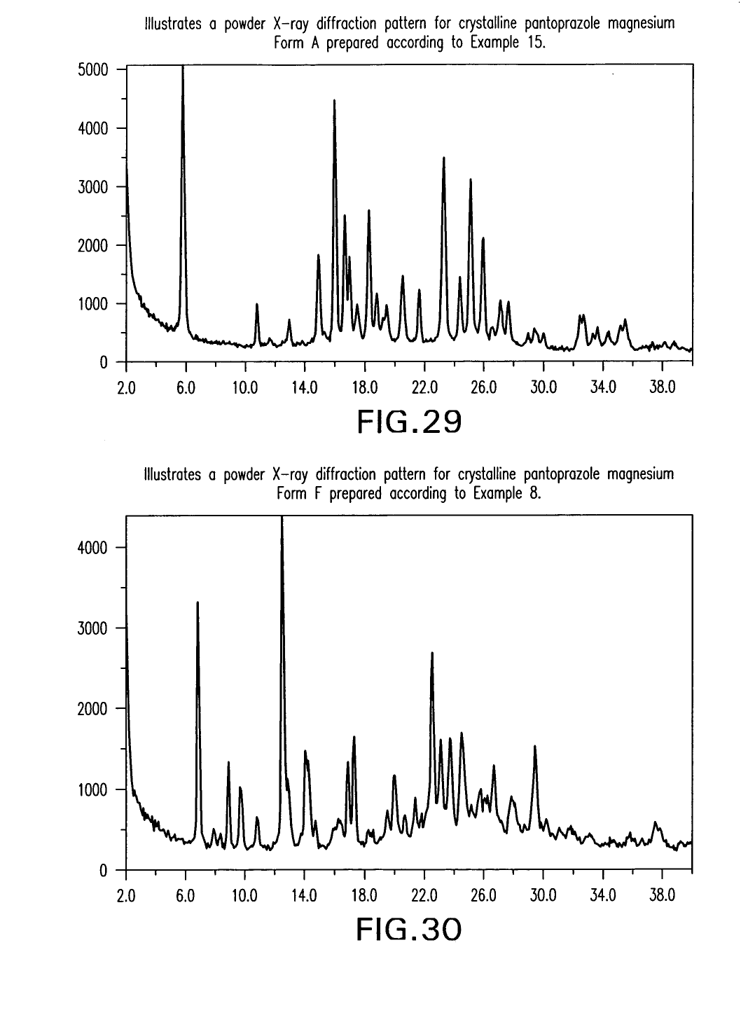

Samples of crystalline pantoprazole magnesium Forms A (prepared according to example 15), E, F, G, and H were each analyzed by light microscope in light oil. The results are depicted in FIGS. 29-33, respectively.

a) Form A

As illustrated by FIG. 29, Form A is made up of plate-shaped particles having a particle size of less than 50 microns.

b) Form E

As illustrated by FIG. 30, Form E is made up of plate-shaped particles having having a particle size of less than 20 microns. Form E has a smaller particle size than Form A, which eliminates the need for milling before use in a pharmaceutical composition. Milling often causes polymorphic transformations.

c) Form F

As illustrated by FIG. 31, Form F is made up of spherical particles. Form F is made up of spherical particles, which are often more flowable than the plate-shaped particles characteristic of Form A. Increased flowability allows for ease in manufacturing the pharmaceutical composition.

d) Form G

As illustrated by FIG. 32, Form G is made up of particles having a particle size of less than 10 microns. Form G has a smaller particle size than Form A, which eliminates the need for milling before use in a pharmaceutical composition.

e) Form H

As illustrated by FIG. 33, Form H is made up of very small particles. Form H has a smaller particle size than Form A, which eliminates the need for milling before use in a pharmaceutical composition.

### Example 19

### Tablet preparation from amorphous Pantoprazole Magnesium, PNT-MG Form C, Form E, Form G and Form H

40 g PNT-Mg (Amorphous form, Form C, Form E, Form G, Form H), 110 mg Mannitol, 15 mg Crospovidone and 15 mg Povidone were measured in a container. The participants in the container were mixed during 5 minutes by VORTEX, obtaining a well mixed blend. The mixture was pressed under a pressure of 2 tons using a round punch (1 cm diameter) during 5 s, obtaining tablet.

1. Crystalline pantoprazole magnesium hemipentahydrate.

2. Crystalline pantoprazole magnesium tetrahydrate.

3. Crystalline pantoprazole magnesium dimethanolate.

4. Amorphous pantoprazole magnesium.

5. Amorphous pantoprazole magnesium of claim 4, which contains no more than about 10% by weight of crystalline pantoprazole magnesium Form A or form C.

6. (canceled)

7. A process for preparing the amorphous pantoprazole magnesium of claim 4 comprising: dissolving pantoprazole magnesium in a solvent selected from methanol and ethanol; and removing the solvent to obtain the amorphous pantoprazole magnesium.

8-9. (canceled)

10. A crystalline form of pantoprazole magnesium characterized by at least one of: a powder X-ray diffraction pattern having peaks at about 6.0, 16.0, 19.0 and 19.6±0.2° 2θ; a powder X-ray diffraction pattern having peaks at about 6.0, 16.0, 19.0, 19.6, and 23.3±0.2° 2θ; a powder X-ray diffraction pattern having peaks at about 16.0, 16.6, 18.3, 19.0 and 19.6±0.2° 2θ; a powder X-ray diffraction pattern substantially as depicted in FIG. 5; a PXRD pattern having peak position differences between the peak at 15.0±0.4° 2θ of about 1.0, 1.6, 3.3, 4.0 and 4.6±0.10° 2θ; an infrared spectrum having bands at about 3275, 2991, 1593, 1428, 1074, 1035 and 827±2 cm⁻¹; an IR spectrum substantially as depicted in FIG. 14.

11-18. (canceled)

19. A process for preparing the crystalline form of pantoprazole magnesium of claim 10 comprising: combining pantoprazole magnesium with methanol, ethanol, methyl-t-butyl ether, ethyl acetate and mixtures thereof to obtain a slurry of the crystalline form of pantoprazole magnesium; heating and isolating the crystalline form of pantoprazole magnesium from the slurry.

20. A crystalline form of pantoprazole magnesium characterized by at least one of: a powder X-ray diffraction pattern having peaks at about 5.6, 13.1, and 16.6±0.2° 2θ a powder X-ray diffraction pattern having peaks at about 5.6, 13.1, 16.6, and 22.3±0.2° 2θ; a powder X-ray diffraction pattern having peaks at about 5.6, 12.5, 13.1, and 16.6±0.2° 2θ; a powder X-ray diffraction pattern substantially as depicted in FIG. 7; an infrared spectrum having bands at about 3532, 1664, 1412, 1388, 1002, and 883±2 cm⁻¹; an IR spectrum substantially as depicted in FIG. 15; a solid state ¹³C NMR spectrum having signals at about 102.5, 118.3, 143.8 and 157.7±0.2 ppm; a SSNMR spectrum substantially as depicted in FIG. 19; a the solid state ¹³C NMR spectrum having chemical shift differences between the signal exhibiting the lowest chemical shift and another in the chemical shift range of 100 to 200 ppm of about 0, 15.8, 41.3 and 55.2±0.1 ppm.

21-30. (canceled)

31. A process for preparing the crystalline form of pantoprazole magnesium of claim 20 comprising:
a) combining pantoprazole free acid, a source of magnesium selected from the group consisting of Mg, Mg(OCH₂CH₃)₂ and Mg(OCH₃)₂, methanol and water to obtain a mixture;
b) heating the mixture;
c) cooling the mixture to precipitate the crystalline form of pantoprazole magnesium; and
d) isolating the precipitated crystalline form of pantoprazole magnesium of claim 0 from the mixture.

32. A crystalline form of pantoprazole magnesium characterized by at least one of: a powder X-ray diffraction pattern having peaks at about 6.9, 8.9, 9.7, 12.4, and 14.0±0.2° 2θ; a powder X-ray diffraction pattern having peaks at about 6.9, 8.9, 9.7, 12.4, 14.0, 16.9, 17.2, and 22.5±0.2° 2θ; a powder X-ray diffraction pattern substantially as depicted in FIG. 8 or 9; an infrared spectrum having bands at about 3657, 2982, 1587, 1408, 1176, 1156, and 1069±2 cm⁻¹; an infrared spectrum substantially as depicted in FIG. 16; a solid state ¹³C NMR spectrum having signals at about 106.1, 142.2, 144.0 and 160.2±0.2 ppm; a SSNMR spectrum substantially as depicted in FIG. 20; a the solid state ¹³C NMR spectrum having chemical shift differences between the signal exhibiting the lowest chemical shift and another in the chemical shift range of 100 to 200 ppm of about 0, 36.1, 37.9 and 54.1±0.1 ppm.

33-40. (canceled)

41. A process for preparing the crystalline form of pantoprazole magnesium of claim 32 comprising:
a) combining pantoprazole free acid, a source of magnesium selected from the group consisting of Mg, Mg(OCH₃)₂ and Mg(OCH₂CH₃)₂, and methanol to obtain a mixture;
b) heating the mixture;
c) cooling the mixture to precipitate the crystalline form; and
d) isolating the precipitated crystalline form of pantoprazole magnesium.

42. (canceled)

43. A process for preparing crystalline pantoprazole magnesium Form A comprising: combining the crystalline form of claim 32 with a solvent selected from water, ethanol, isopropanol, and mixtures of water and a C₁-C₄ alcohol to obtain a slurry of the crystalline pantoprazole magnesium Form A.

44. A process for preparing crystalline pantoprazole magnesium Form A comprising: dissolving the crystalline form of claim 32 in methanol to form a solution; and adding water to the solution to precipitate the crystalline pantoprazole magnesium Form A.

45. A process for preparing crystalline pantoprazole magnesium Form A comprising: dissolving pantoprazole magnesium in methanol to form a solution; and adding water to the solution to precipitate the crystalline pantoprazole magnesium Form A.

46. A crystalline form of pantoprazole magnesium characterized by a PXRD pattern having peaks at about 7.2, 12.9, 13.8, and 17.0±0.2° 2θ; a PXRD pattern having peaks at about 7.2, 12.9, 13.8, 14.6, 17.0, 21.7, and 22.7±0.2° 2θ; a PXRD pattern substantially as depicted in FIG. 10; an infrared spectrum having bands at about 3657, 2976, 1647, 1420, 1405, 1179, and 1066±2 cm⁻¹; an infrared spectrum substantially as depicted in FIG. 17; a solid state ¹³C NMR spectrum having signals at about 108.2, 113.4, 143.6, 157.1±0.2 ppm; a solid state ¹³C NMR spectrum having chemical shift differences between the signal exhibiting the lowest chemical shift and another in the chemical shift range of 100 to 200 ppm of about 0, 5.2, 35.4 and 48.9±0.1 ppm; a solid state ¹³C NMR spectrum substantially as depicted in FIG. 21.

47-55. (canceled)

56. A process for preparing the crystalline form of pantoprazole magnesium of claim 43 comprising exposing a crystalline form of pantoprazole magnesium characterized by at least one of: a powder X-ray diffraction pattern having peaks at about 6.9, 8.9, 9.7, 12.4, and 14.0±0.2° 2θ; a powder X-ray diffraction pattern having peaks at about 6.9, 8.9, 9.7, 12.4, 14.0, 16.9, 17.2, and 22.5±0.2° 2θ; a powder X-ray diffraction pattern substantially as depicted in FIG. 8 or 9; an infrared spectrum having bands at about 3657, 2982, 1587, 1408, 1176, 1156, and 1069±2 cm⁻¹; or a solid state ¹³C NMR spectrum having signals at about 106.1, 142.2, 144.0 and 160.2±0.2 ppm to about 0-20% relative humidity at about room temperature.

57. A crystalline form of pantoprazole magnesium-characterized by at least one of: a PXRD pattern having peaks at about 6.8, 9.0, 9.5, 12.9, and 13.8±0.2° 2θ; a PXRD pattern having peaks at about 6.8, 9.0, 9.5, 12.9, 13.8, 17.2 and 22.6±0.2° 2θ; a PXRD pattern substantially as depicted in FIG. 11; an infrared spectrum having bands at about 3651, 1588, 1424, 1410, and 468±2 cm⁻¹; an infrared spectrum substantially as depicted in FIG. 18; a solid state ¹³C NMR spectrum having signals at about 107.6, 144.1, 157.8, 159.5±0.2 ppm; a solid state ¹³C NMR spectrum has chemical shift differences between the signal exhibiting the lowest chemical shift and another in the chemical shift range of 100 to 200 ppm of about 0, 36.5, 50.2 and 51.9±0.1 ppm; a solid state ¹³C NMR spectrum substantially as depicted in FIG. 22.

58-66. (canceled)

67. A process for preparing the crystalline form of pantoprazole magnesium of claim 57 comprising exposing a crystalline form of pantoprazole magnesium characterized by at least one of: a powder X-ray diffraction pattern having peaks at about 6.9, 8.9, 9.7, 12.4, and 14.0±0.2° 2θ; a powder X-ray diffraction pattern having peaks at about 6.9, 8.9, 9.7, 12.4, 14.0, 16.9, 17.2, and 22.5±0.2° 2θ; a powder X-ray diffraction pattern substantially as depicted in FIG. 8 or 9; an infrared spectrum having bands at about 3657, 2982, 1587, 1408, 1176, 1156, and 1069±2 cm⁻¹; or a solid state ¹³C NMR spectrum having signals at about 106.1, 142.2, 144.0 and 160.2±0.2 ppm to about 40-100% relative humidity at about room temperature.

68. A pharmaceutical composition comprising
a) a therapeutically effective amount of at least one of:
i) amorphous pantoprazole magnesium
ii) amorphous pantoprazole magnesium having less than about 10% by weight of crystalline pantoprazole magnesium Form A or form C;
iii) a crystalline form characterized by at least one of: a powder X-ray diffraction pattern having peaks at about 6.0, 16.0, 19.0 and 19.6±0.2° 2θ; a powder X-ray diffraction pattern having peaks at about 6.0, 16.0, 19.0, 19.6, 23.3 and +0.2° 2θ; a powder X-ray diffraction pattern having peaks at about 16.0, 16.6, 18.3, 19.0 and 19.6±0.2° 2θ; a powder X-ray diffraction pattern substantially as depicted in FIG. 5; a PXRD pattern having peak position differences between the peak at 15.0±0.4° 2θ of about 1.0, 1.6, 3.3, 4.0 and 4.6±0.10° 2θ; an infrared spectrum having bands at about 3275, 2991, 1593, 1428, 1074, 1035 and 827±2 cm⁻¹; an IR spectrum substantially as depicted in FIG. 14;
iv) a crystalline form of pantoprazole magnesium characterized by at least one of: a powder X-ray diffraction pattern having peaks at about 5.6, 13.1, and 16.6±0.2° 2θ a powder X-ray diffraction pattern having peaks at about 5.6, 13.1, 16.6, and 22.3±0.2° 2θ; a powder X-ray diffraction pattern having peaks at about 5.6, 12.5, 13.1, and 16.6±0.2° 2θ; a powder X-ray diffraction pattern substantially as depicted in FIG. 7; an infrared spectrum having bands at about 3532, 1664, 1412, 1388, 1002, and 883±2 cm⁻¹; an IR spectrum substantially as depicted in FIG. 15; a solid state ¹³C NMR spectrum having signals at about 102.5, 118.3, 143.8 and 157.7±0.2 ppm; a SSNMR spectrum substantially as depicted in FIG. 19; a solid state ¹³C NMR spectrum having chemical shift differences between the signal exhibiting the lowest chemical shift and another in the chemical shift range of 100 to 200 ppm of about 0, 15.8, 41.3 and 55.2±0.1 ppm;
v) a crystalline form of pantoprazole magnesium characterized by a PXRD pattern having peaks at about 7.2, 12.9, 13.8, and 17.0±0.2° 2θ; a PXRD pattern having peaks at about 7.2, 12.9, 13.8, 14.6, 17.0, 21.7, and 22.7±0.2° 2θ; a PXRD pattern substantially as depicted in FIG. 10; an infrared spectrum having bands at about 3657, 2976, 1647, 1420, 1405, 1179, and 1066±2 cm⁻¹; an infrared spectrum substantially as depicted in FIG. 17; a solid state ¹³C NMR spectrum having signals at about 108.2, 113.4, 143.6, 157.1±0.2 ppm; a solid state ¹³C NMR spectrum having chemical shift differences between the signal exhibiting the lowest chemical shift and another in the chemical shift range of 100 to 200 ppm of about 0, 5.2, 35.4 and 48.9±0.1 ppm; a solid state ¹³C NMR spectrum substantially as depicted in FIG. 21;
vi) A crystalline form of pantoprazole magnesium characterized by at least one of: a PXRD pattern having peaks at about 6.8, 9.0, 9.5, 12.9, and 13.8±0.2° 2θ; a PXRD pattern having peaks at about 6.8, 9.0, 9.5, 12.9, 13.8, 17.2 and 22.6±0.2° 2θ; a PXRD pattern substantially as depicted in FIG. 11; an infrared spectrum having bands at about 3651, 1588, 1424, 1410, and 468±2 cm⁻¹; an infrared spectrum substantially as depicted in FIG. 18; a solid state ¹³C NMR spectrum having signals at about 107.6, 144.1, 157.8, 159.5±0.2 ppm; a solid state ¹³C NMR spectrum has chemical shift differences between the signal exhibiting the lowest chemical shift and another in the chemical shift range of 100 to 200 ppm of about 0, 36.5, 50.2 and 51.9±0.1 ppm., a solid state ¹³C NMR spectrum substantially as depicted in FIG. 22; and
b) at least one pharmaceutically acceptable excipient.

69. A method of inhibiting gastric acid secretion comprising administering the pharmaceutical composition of claim 68 to a patient in need thereof.
